SEQUENCE LISTING (12 sequences)

SEQ ID NO: 1 - protein, 4 aa, Artificial Sequence
  carboxyl-terminal endoplasmic reticulum retrieval signal
KDEL

SEQ ID NO: 2 - DNA, 33 bp, Artificial Sequence
  primer_bind: sFvA primer
gctgcccaaccagccatggcggacattgtgatg

SEQ ID NO: 3 - DNA, 30 bp, Artificial Sequence
  primer_bind: sFvD primer
gccaccgccaccactagttgaggagactgt

SEQ ID NO: 4 - DNA, 44 bp, Artificial Sequence
  primer_bind: sFvB primer
aagccaggctccggcgaaggcagcaccaaaggcgaagtgaagtt

SEQ ID NO: 5 - DNA, 48 bp, Artificial Sequence
  primer_bind: sFvC primer
gccggagcctggcttgccgctgccgctggtggagcctttgatcaccag

SEQ ID NO: 6 - protein, 5 aa, Artificial Sequence
  peptide tether between the genes encoding scFvZME-018 and gelonin
GGGGS

SEQ ID NO: 7 - DNA, 37 bp, Artificial Sequence
  primer_bind: primer 3
ccggagccaccgccaccgctagctgaggagactgtga

SEQ ID NO: 8 - DNA, 33 bp, Artificial Sequence
  primer_bind: primer 2
ggtggcggtggctccggtctagataccgttagc

SEQ ID NO: 9 - DNA, 49 bp, Artificial Sequence
  primer_bind: primer 4
gccgcaagcttaatagttacagctcgtctttctcgaggaatttcagcag

SEQ ID NO: 10 - protein, 18 aa, Artificial Sequence
  peptide tether between two antibody variable regions
GSTSGSGKPGSGEGSTKG

SEQ ID NO: 11 - DNA, 1542 bp, Artificial Sequence
  nucleotide sequence of the encoding the scFvZME-gelonin immunotioxin
ccgatggcggacattgtgatgaccgagtctcaaaaattcatgtccacatcagtaggagacagggtcagggtgacctgcaaggcgagtcagaatgtggatactaatgtagcctggtatcaacaaaaaccagggcaatctcctcaaccactgcttttctccgcatcctaccgttacagtggagtgcctcgtcgtttcacaggcagtggatctgggacagatttcactctcaccatcagcaatgtgcagtctgaagacttggcagagtatttctctgagcaatctaacagttatcctctgacgttcggtggaggcaccaagctgcagatcaaagggtccacgagcggcagcggtaagccagggtccggcgaaggcagcaccaaaggcgaagtgaaggttgaggagtctggaggaggcttggtgcaacctggaggatccatgaaactctcctctgttgtctctggattcactttcggtaattactggatgaactgggtccgccagtctccagagaaggggcttgagtggattgcagaaattagattgaaatcgaataattttgcaagatattatgctgagtctgtgaaagggaggttcaccatctcaagagatgattccaaaagtagtgtctacctggaaatgatcaacctaagagctgaagatactggcatttattactctacgacttatggtaactacgttgggcactacttggagcactggggccaaggcaccactctcaccgtctcctcaggtggcggtggctccggtctagataccgttagcttcagcaccaaaggcgcgacctatatcacctacgttaatttcctgaacgaactgcgtgttaaactgaaaccggaaggtaacagccatggcatcccgctgctgcgtaaaggtgatcaccccggtaaatgcttcgtgctggtggcactgagcaacgataacggtcagctggcagaaatcgcaatcgatgttaccagcgtgtacgtagttggctatcaggtgcgtaaccgcagctacttcttcaaagatgctccggatgcagcgtacgaaggcctgttcaaaaagacgatcaaaaacccgctgctgttcggtggcaaaactcgtctgcacttcggtggcagctatccgagcctggaaggcgaaaaagcgtaccgcgaaactaccgatctgggtatcgaaccgctgcgcatcggcatcaaaaaactggaggaaaacgcgatcgacaactacaaaccgaccgaaatcgcgacctctctgctggttgtgatccagatggtgagcgaagcggcacgtttcaccttcatcgaaaaccagattcgtaacaacttccagcagcgtatccgtccggcgaacaacacgatctctctggaaaacaaatggggcaaactgagcttccagatccgtaccagcggtgcgaacggtatgttcagcgaagcggtggaactggaacgcgcgaacggcaaaaaatactacgtgactgcggtggatcaggtgaaagcgaaaatcgcactgctgaaattcctcgagaaagacgagctgtaa

SEQ ID NO: 12 - protein, 513 aa, Artificial Sequence
  predicted amino acid sequence of the scFvZME-gelonin immunotioxin
PMADIVMTQSQKFMSTSVGDRVSVTCKASQNVDTKVAWYQQKPGQSPQPLLFSASYRYSGVPRRFTGSGSGTDFTLTISNVQSEDLAEYFCQQYNSYPLTFGGGTKLGIKGSTSGSGKPGSGEGSTKGEVKVEESGGGLVQPGGSMKLSCVVSGFTFGNYWMNWVRQSPEKGLEWIAEIRLKSNNFARYYAESVKGRFTISRDDSKSSVYLEMINLRAEDTGIYYCTSYGNYVGHYLDHWGQGTTLTVSSGGGGSGLDTVSFSTKGATYITYVNFLNELRVKLKPEGNSHGIPLLRKGDDPGKCFVLVALSNDNGQLAEIAIDVTSVYVVGYQVRDRSYFFKDAPDAAYEGLFKNTIKNPLLFGGKTRLHFGGSYPSLEGEKAYRQTTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFLEKDEL

(19) United States
(12) Patent Application Publication
(10) Pub. No.: US 2005/0214307 A1
(43) Pub. Date: Sep. 29, 2005

(54) Antibody conjugates for treatment of neoplastic disease

(75) Inventor: Michael G. Rosenblum, Sugar Land, TX (US)

Correspondence Address: David L. Parker;FULBRIGHT & JAWORSKI L.L.P., Suite 2400, 600 Congress Avenue, Austin, TX, 78701, US

(73) Assignee: Research Development Foundation

(21) Appl. No.: 10/926,731

(22) Filed: Aug. 26, 2004

Related U.S. Application Data
(63) Continuation of application No. 08/406,178, filed on Mar. 17, 1995, now abandoned.
Application No. 08/406,178 is a continuation-in-part of application No. 08/252,327, filed on Jun. 1, 1994, now abandoned.
Application No. 08/252,327 is a continuation-in-part of application No. 08/119,900, filed on Sep. 10, 1993, now abandoned.
Application No. 08/119,900 is a continuation of application No. 07/950,780, filed on Sep. 24, 1992, now abandoned.
Application No. 07/950,780 is a continuation of application No. 07/510,923, filed on Apr. 19, 1990, now abandoned.

(51) Int. Cl. A61K 39/395; C07K 16/46

(52) U.S. Cl. 424/178.1; 530/391.1

(57) ABSTRACT

Immunoconjugates of an antibody to a 240 kD melanoma tumor associated antigen were prepared. Cytotoxic immunoconjugates such as ZME-018 antibody conjugate are useful for treating proliferative cell diseases such as melanoma as well as other tumors which bear the ZME-018 antigen. Detectably labeled compositions for diagnosis of such diseases are also disclosed.

### CROSS REFERENCE TO RELATED APPLICATION

This application is a continuation in part of U.S. Ser. No. 08/252,327, filed Jun. 1, 1994, which is a continuation in part of U.S. Ser. No. 08/119,900, filed Sep. 10, 1993, which is a continuation of U.S. Ser. No. 07/950,780, filed Sep. 24, 1992, now abandoned, which is a continuation of U.S. Ser. No. 07/510,923, filed Apr. 19, 1990, now abandoned.

### BACKGROUND OF THE INVENTION

1. Field of the Invention

The present invention relates generally to the field of immunoconjugates. More particularly, the present invention relates to the use of immunoconjugates in the treatment of cancer.

2. Description of the Related Art

Melanoma, the most virulent of skin cancers, is a highly metastatic disease affecting both sexes and is almost uniformly fatal within five years of diagnosis. Surgical removal of localized malignancies has proven effective only when the disease has not spread beyond the primary lesion. Once the disease has spread, the surgical procedures must be supplemented with other more general procedures to eradicate the diseased or malignant cells. Most of the commonly utilized supplementary procedures such as irradiation or chemotherapy are not localized to the tumor cells and, although they have a proportionally greater destructive effect on malignant cells, often affect normal cells to some extent.

Many tumors express antigens or antigenic determinants which are either expressed very weakly or not expressed at all by normal cells. Some tumor cells express antigens which are expressed by embryonic cell types but are not expressed by normal cells of a mature animal. These abnormally expressed antigens are known as tumor-associated antigens. These antigens are specific in that while a particular antigen may be expressed by more than one tumor, it is usually expressed by all or most cells of the particular tumors which express it. A tumor cell may express one or more than one tumor-associated antigen. These tumor-associated antigens may be expressed on the surface of the cell (cell surface antigen), may be secreted by the tumor cell (secreted antigens) or may remain inside the cell (intracellular antigen).

The presence of these tumor-associated antigens has been utilized to detect, diagnose and localize the tumor. In some cases the presence of the tumor-associated antigens on the tumor cells has allowed the targeting of specific drugs and other treatment means specifically to the tumor cells.

The cytotoxic agents frequently utilized for antibody conjugates primarily fall into three classes of agents: toxins, radionuclides and chemotherapeutic agents. Antibody conjugates with each of these types of agents offer substantial promise as therapeutic agents but present some unique problems as well (Frankel, et al. Ann. Rev. Med. 37: 125-142 (1986); Reimann et al., J. Clin. Invest. 82(1): 129-138 (1988).).

Gelonin is a glycoprotein (M.W. approximately 29-30,000 Kd) ribosomal-inactivating plant toxin purified from the seeds of Gelonium multiforum. Other members of this class of ribosomal-inactivating plant toxins are the chains of abrin, ricin and modeccin. Gelonin, like abrin and ricin, inhibits protein synthesis by damaging the 60S sub-unit of mammalian ribosomes. Although the A chain of ricin (RTA) has been popular for use in immunotoxins, gelonin appears to be more stable to chemical and physical treatment than RTA. Furthermore, gelonin itself does not bind to cells and is, therefore, non-toxic (except in high concentrations) and is safe to manipulate in the laboratory. The inactivation of ribosomes is irreversible, does not appear to involve co-factors and occurs with an efficiency which suggests that gelonin acts enzymatically.

Numerous prior workers have suggested or reported linking cytotoxic agents to antibodies to make “immunotoxins.” Of particular interest have been immunotoxins of monoclonal antibodies conjugated to the enzymatically active portions (A chains) of toxins of bacterial or plant origin such as ricin or abrin .

Previous studies have described a number of antibody-toxin conjugates containing gelonin (Lambert et al., J. Biol. Chem. 260: 12035-12038 (1985); Thorpe et al., Eur. J. Biochem 116: 447-454 (1981); Singh et al., J. Biol. Chem. 264: 3089-95 (1989); Scott et al., J. Natl. Cancer Inst. 79: 1163-72 (1987); Tedder et al., J. Immunol. 137(4): 1387-91 (1986)). Recently Ozawa, et al. (Int. J. Cancer 43: 152-157) have constructed a gelonin immunotoxin comprised of antibody B467 which binds to the cellular receptor for epidermal growth factor (EGF). This B467-gelonin conjugate was highly cytotoxic for EGF receptor expressing cells but was non-cytotoxic for receptor-deficient cells. Sivam, et al. (Cancer Research 47: 3169-3173 (1987)) have made a conjugate of the antimelanoma antibody 9.2.2.7 with gelonin and compared in vitro and in vivo cytotoxic activity with a 9.2.2.7 conjugate of abrin and ricin A chain. However, these studies demonstrated that gelonin conjugates demonstrated were not toxic in in vivo experiments up to 2 mg total antibody dose/mouse and that multiple I.V. administration of gelonin immunotoxin was required to significantly retard the growth of an established subcutaneous human tumor xenograft in nude mice.

The prior art remains deficient in the lack of effective immunotoxins for the treatment of different carcinomas. The present invention fulfils the longstanding need and desire in the art.

### SUMMARY OF THE INVENTION

The present invention provides immunoconjugates of an antibody (herein designated ZME-018) which recognizes the GP 240 antigen on melanoma cancer cells. One of the antibodies (225.28S) discussed by Wilson et al. (Wilson, et al., Int. J. Cancer 28: 293 (1981)) recognizes this melanoma membrane antigen. This antigen is identified therein by the designation GP 240. The antibody 225.28S which binds the GP 240 antigen has been designated and is further referred to herein as ZME-018. In one embodiment, the antibody is coupled with a toxin selected from the group consisting of gelonin, ricin A chain and abrin A chain. In another embodiment the ZME antibody may be coupled with a cytocidal drug such as adriamycin or a biological response modifier such as a lymphokine or cytokine. In another embodiment the antibody may be labeled with a detectable label such as a radiolabel, a chemiluminescer, a fluorescer, or an enzyme label. The cytocidal immunoconjugates are useful to treat and prevent recurrence of tumor-associated GP 240-bearing tumors by administration of these cytocidal immunoconjugates to an individual in need of such treatment. The detectably labeled ZME immunoconjugates are useful for diagnosis and localization of tumors by techniques known to those in the art. These labelled immunoconjugates are also useful to assay for the presence of the GP 240 antigen in biological specimens and for localizing the tumor site in vivo by means known to those of skill in the art.

In another embodiment, there is provided an immunotoxin, comprising: a single chain antibody directed against the 240 kD antigen of gp240; and a cytotoxic moiety.

One of the objects of the present invention is to provide a cytotoxic composition which would specifically bind to and kill tumor cells. Particularly, it is an object of the present invention to provide a cytotoxic composition which would specifically bind to and kill tumor cells which express the GP 240 antigen as described above. Antibody ZME-018 was prepared at Hybritech, Inc. using salt fractionation and DEAE chromatography and was judged homogenous by SDS PAGE (Wilson et al., Int. J. Cancer 28: 293-300 (1981)). Another aspect of the invention concerns a method of killing human melanoma cells, or any other tumor cells expressing the ZME (GP 240) antigen, by contacting the cells with a cytocidally effective amount of an immunotoxin. The present also demonstrates the in vivo tissue distribution, and pharmacokinetics of ZME-gelonin immunoconjugate. Finally, we have also illustrated therapeutic effects of ZME-gelonin conjugate against well-established human melanoma (A375-M) xenografts in nude mice and increased survival of mice treated with ZME-gelonin compared to saline control in highly metastatic AAB-527 melanoma model.

It is a further object of the present invention to provide such a composition which would be toxic to tumor cells but cause minimal injury to normal tissue. Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 demonstrates the coupling and purification schema for ZME-gelonin.

FIG. 2 demonstrates the purification of ZME- gelonin by S-300 gel permeation chromatography.

FIG. 3 demonstrates the elution profile of the Cibachron-Blue sepharose column after the high-molecular weight material from S-300 chromatography was applied and eluted with a linear salt gradient (0-300 mM Nacl). Two protein peaks were demonstrated: a flow-through peak (fractions 14-20) and a bound peak eluted with high salt (fractions 44-75).

FIG. 4 demonstrates the electrophoretic pattern of gelonin and ZME gelonin conjugate.

FIG. 5 demonstrates comparative ELISA Assay data of ZME (open circles) and ZME gelonin (closed circles).

FIG. 6 demonstrates the cytotoxicity of ZME-gelonin and free gelonin on log-phase AAB-527 cells after 72 hour exposure.

FIG. 7 demonstrates the cytotoxicity of ZME-gelonin and free gelonin on log-phase AAB-527 cells.

FIG. 8 demonstrates the cytotoxicity of ZME-gelonin on antigen positive target melanoma cells (AAB-527) and antigen negative T-24 cells in culture.

FIG. 9 demonstrates the influence of free antibody on ZME-gelonin cytotoxicity.

FIG. 10 demonstrates the effect of IFN-α, IFN-γ and TNF on ZME-gelonin cytotoxicity. Closed circles show the dose-response for ZME-gelonin alone. Open diamonds show the dose-response for ZME-gelonin plus IFN-γ. Open triangles show the dose-response for ZME-gelonin in the presence of a fixed amount of TNF-α. Closed circles with dotted lines show ZME-gelonin dose-response curve in the presence of a fixed amount of IFN-α.

FIG. 11 demonstrates the effect of ZME-gelonin on antigen positive (A-375, closed circles) and antigen negative (CEM, open squares) cells in a human tumor stem cell assay.

FIG. 12 demonstrates the cytotoxic effect of ZME-gelonin on stem cell survival of different lines obtained from fresh biopsy specimens of 4 different patients.

FIG. 13 demonstrates the tissue distribution of ZME antibody and ZME-gelonin conjugate in nude mice bearing human melanoma zenografts.

FIG. 14 shows the tissue distribution of ¹²⁵I-labeled Mab ZME-018, ZME-gelonin (antigen positive Mab & immunoconjugate) and 15A8-gelonin (antigen negative immunotoxin) 24 hours after the injection. The results are expressed as tissue:blood ratio. Both Mab ZME and ZME-gelonin immunotoxin localize well within the tumors.

FIG. 15 shows the tissue distribution of radiolabeled Mab ZME-, ZME-gelonin, and 15A8-gelonin, 72 hours after the injections demonstrating a uniform distribution of irrelevant immunotoxin in all the organs; whereas, both antigen specific Mab and immunotoxin (ZME and ZME-gelonin) localizes specifically in the tumors.

FIG. 16 shows the plasma clearance of Mab ZME and ZME-gelonin immunotoxin. The Figure shows the data points and best-fit least square line through the datapoints. Both curves are biphasic with immunotoxin clearance only slightly faster than Mab ZME-018 itself.

FIG. 17 shows the growth suppression of rapdily progressing well established human melanoma (A375-M) tumors in athymic (nu/nu) mice. Tumor cells were innoculated subcutaneously and saline; gelonin; Mab ZME-018; and ZME-gelonin as injected (i.v.) on the days 7, 11, 14, 19, 21, and 25 and continued until day 47.

FIG. 18 shows the scheme for the PCR-based construction of the gene encoding scFvZME-018. The procedure is a modification of Davis et al., Biotechnology, 9:165-169 (1991). Nco I and Spe I restriction sites were incorporated into the sequences of the primer 2 and 3 as indicated by the filled boxes at the 5′ ends of each primer.

FIG. 19 shows a western blot analysis of scFvZME-018 clones isolated from periplasmic lysates. Samples were separated by 12% SDS-PAGE, transferred to nitrocellulose filters and detected with a goat anti-mouse kappa primary antibody followed by a horseradish peroxidase conjugate of a swine anti-goat IgG secondary antibody. The signal was developed using the Amersham ECL system with an exposure of 5 minutes. Lane 1: prestained molecular weight standards; Lane 2: scFvSME-018 clone 1; Lane 3: negative control; Lane 4: positive control 15A8 antibody; Lanes 5-8: scFvZME-018 clones 2-5.

FIG. 20 shows a binding analysis of 34 individual scFvZME-018-gelonin immunotoxin (IT) clones. Periplasmic extracts of individually expressed immunotoxins were added to wells of a 96-well ELISA plate coated with either A375M melanoma cells or mouse anti-gelonin antibody 13A3. Bound IT was detected with a rabbit anti-gelonin polyclonal followed by addition of a horseradish peroxidase (HRPO) conjugate of a goat anti-rabbit IgG secondary antibody. Signals were developed for 20 minutes with the HRPO substrate ABTS and quantitated at 405 nm.

FIG. 21 shows a western blot analysis of scFvZME-018-gelonin immunotoxin clones isolated from small scale (5 ml) periplasmic lyzates. Samples were separated by 12% SDS-PAGE, transferred to nitrocellulose filters and detected with a rabbit anti-gelonin primary antibody followed by a horseradish peroxidase conjugate of a goat anti-rabbit IgG secondary antibody. The signal was developed using the Amersham ECL system with an exposure of 5 minutes. Lane 1: prestained molecular weight standards; Lane 2-7: scFvSME-018 clones 1-5; Lane 8: wild type gelonin; Lane 9: gelonin with a C-terminal KDEL sequence; Lanes 10: gelonin positive control 20 ng.

### DETAILED DESCRIPTION OF THE INVENTION

As used herein the term “monoclonal antibody” means an antibody composition having a homogeneous antibody population. It is not intended to be limited as regards the source of the antibody or the manner in which it is made.

Melanoma cells express a 240 kDa (gp 240) antigen on their cell surface. Antibody ZME-018 (from Hybritech, Inc.) is a murine monoclonal antibody IgG₂ₐ recognizing a 240 Kd glycoprotein present on most human melanoma cells. Monoclonal antibodies of the IgG₁, IgG₂ₐ and IgG_{2b} isotypes which recognize an epitope of this 240 kDa antigen may be produced. This 240 Kd epitope of the ZME antigen is designated the ZME epitope. Thus, all antibodies which recognize this ZME epitope are functionally equivalent.

These representative hybridoma cultures whose cells secrete antibody of the same idiotype, i.e., all recognize the ZME epitope, have been deposited at the American Type Culture Collection of 12301 Parklawn Drive, Rockville, Md. 20852 (“ATCC”) and have been assigned the Accession number 11009.

The present invention provides a composition of matter comprising a conjugate of a ZME antibody and a cytotoxic moiety. Generally, the moiety is selected from the group consisting of a toxin, a cytocidal, a cytostatic drug and a biological response modifier. Most preferably, the moiety is gelonin.

The present invention is also directed to a method of treating proliferative cell diseases characterized by tumors expressing an antigen to which ZME antibody binds, comprising administration of a cytocidally effect dose of the composition of claim 1 to an individual in need of said treatment. In another embodiment, the present invention involves a method of treating melanoma comprising administration of a gelonin coupled monoclonal antibody directed to ZME antigen to an individual having melanoma. In yet another embodiment of the present invention, there is provided a method of preventing recurrence of melanoma tumors comprising administration of gelonin conjugated monoclonal antibody ZME to an individual diagnosed as having a tumor bearing ZME tumor associated antigen.

In still yet another embodiment of the present invention, there is provided a method of enhancing the cytotoxic activity of immunotoxins comprising administration of a biological response modifier prior to the administration of an immunotoxin. Generally. the immunotoxin is selected from the group consisting of a gelonin conjugated monoclonal antibody, a ricin conjugated antibody and a TNF conjugated antibody. Preferably, the antibody is selected from the group consisting of an antibody directed against a cell surface antigen of melanoma cells, a cell surface antigen of breast carcinoma cells, and a cell surface antigen of cervical cancer cells. Most preferably, the antibody is ZME-018. Most preferably, the biological response modifier is selected from the group consisting of IFNα and TNFα.

The present invention is also directed to a method of treating proliferative cell diseases characterized by tumors expressing an antigen to which ZME antibody binds, comprising administration of a cytocidally effect dose of the composition of claim 3 to an individual in need of said treatment.

The present invention is also directed to an immunotoxin, comprising: a single chain antibody directed against the 240 kD antigen of gp240; and a cytotoxic moiety. Preferably, the single chain antibody is a single chain ZME-018 antibody. Preferably, the cytotoxic moiety is gelonin. Most preferably, the immunotoxin is recombinantly produced.

Monoclonal antibodies may be made by methods known to those of skill in the art. The characterization of and procedure for making the hybridoma cell cultures which produce these antibodies is described in detail. (Wilson et al., Int. J. Cancer 28:293 (1981); Imai et al, Transplant Proc. 12:380-383 (1980)). Briefly, hybridomas were constructed with the murine myeloma cell line Sp2/0-Ag-14 and splenocytes from mice immunized with the melanoma cell line M21 as described by Imai et al. The hybridomas secreting the monoclonal antibody (MoAb) 225.28S and 465.12 have been subcloned and are propagated in vitro and in vivo. Both monoclonal antibodies are of the IgG₂ₐ subclass and were purified from mouse ascites fluid by absorption/elution from protein A Sepharose 4B (Pharmacia, Piscataway, N.J., USA) prior to their use.

Hybridomas producing antibodies which reacted with human melanoma cells but not with normal human cells were further characterized. The antibodies produced by the ZME cell line and hybridoma producing functionally equivalent antibodies reacted with the ZME antigen on human melanoma cells. They also reacted with 70-80% of randomly-obtained melanomas tested, and exhibited no reaction to various tissues as summarized on TABLE I in Example 4.

As used herein with respect to the exemplified murine monoclonal anti-human melanoma antibodies, the term “functional equivalent” means a monoclonal antibody that: (1) crossblocks an exemplified monoclonal antibody; (b) binds selectively to cells expressing the ZME antigen such as human melanoma cells; (c) has a G or M isotype; (d) binds to the ZME antigen as determined by immunoprecipitation or sandwich immunoassay; and (e) when conjugated to gelonin, exhibits a tissue culture inhibitory dose (TCID) of at least 50% against at least one of the AAB-527, or A375 cell lines when used at a dose of 80-100 units per ml.

Antibody ZME was conjugated to gelonin using N-succinimidyl-3-(2-pyridyldithio)propionate (SPDP) or 2-iminothiolane (IT) as a coupling agent. The conjugates were tested against AAB-527 and A375 cells in a 72-hour tissue culture assay. The antibody conjugates exhibited acceptable antiproliferative activity (TCID 50% of less than 100 units ml) against both of these cell lines. Further details of the characterization of the antibodies are provided in the examples below.

Biological response modifiers which may be coupled to the ZME antibody and used in the present invention include, but are not limited to, lymphokines and cytokines such as IL-1, IL-2, interferons, TNF, LT, TGF-β, and IL- 6. These biological response modifiers have a variety of effects on tumor cells including increased tumor cell killing by direct action and by increased host defense mediated processes. Conjugation of antibody ZME to biological response modifiers will allow selective localization within tumors and, hence, improved antiproliferative effects while suppressing non-specific effects leading to toxicity of non-target cells.

Cytotoxic drugs which are useful in the present invention include, but are not limited to, adriamycin (and derivatives thereof), cis-platinum complex (and derivatives thereof), bleomycin and methotrexate (and derivatives thereof). These cytotoxic drugs are sometimes useful for clinical management of recurrent tumors and particularly melanoma, but their use is complicated by severe side effects and damage caused to non-target cells. Antibody ZME may serve as a useful carrier of such drugs providing an efficient means of both delivery to the tumor and enhanced entry into the tumor cells themselves.

Conjugates of the monoclonal antibody may be made using a variety of bifunctional protein coupling agents. Examples of such reagents are SPDP, IT, bifunctional derivatives of imidoesters such as dimethyl adipimidate. HCl, active esters such as disuccinimidyl suberate, aldehydes such as glutaraldehyde, bis-azido compounds such as bis(p-azidobenzoyl) hexanediamine, bis-diazonium derivatives such as bis-(p-diazoniumbenzoyl)-ethylenediamine, diisocyanates such as tolylene 2,6-diisocyanate, and bis-active fluorine compounds such as a 1,5-difluoro-2,4-dinitrobenzene.

When used to kill human melanoma cells in vitro for therapeutic or for diagnostic purposes, the conjugates will typically be added to the cell culture medium at a concentration of at least about 10 nM. The formulation and mode of administration for in vitro use are not critical. Aqueous formulations that are compatible with the culture or perfusion medium will normally be used.

Cytotoxic radiopharmaceuticals for diagnosing and treating tumors carrying the ZME antigen such as melanoma may be made by conjugating high linear energy transfer (LET) emitting isotopes to the antibodies. The term “cytotoxic moiety” as used herein is intended to include such isotopes.

The labels that are used in making labeled versions of the antibodies include moieties that may be detected directly, such as fluorochromes and radiolabels as well as moieties, such as enzymes, that must be reacted or derivatized to be detected. Examples of such labels are ³²P, ¹²⁵I, ³H, ¹⁴C, fluorescein and its derivatives, rhodamine and its derivatives, dansyl, umbelliferone, luciferia, 2,3-dihydrophthalzainediones, horseradish peroxidase, alkaline phosphatase, lysozyme, and glucose-6-phosphate dehydrogenase. The antibodies may be tagged with such labels by known methods. For instance, coupling agents such as aldehydes, carbodiimides, dimaleimide, imidates, succinimides, bis-diazotized benzadine and the like may be used to couple the antibodies with the above-described fluorescent, chemiluminescent, and enzyme labels.

The antibodies and labeled antibodies may be used in a variety of immunoimaging or immunoassay procedures to detect the presence of tumors expressing the ZME antigen such as melanoma in a patient or monitor the status of such cancer in a patient already diagnosed to have it. When used to monitor the status of a cancer a quantitative immunoassay procedure may be used. Such monitoring assays are carried out periodically and the results compared to determine whether the patient's tumor burden has increased or decreased. Common assay techniques that may be used include direct and indirect assays. Direct assays involve incubating a tissue sample or cells from the patient with a labeled antibody. If the sample ZME antigen bearing cells includes melanoma cells, the labeled antibody will bind to those cells. After washing the tissue or cells to remove unbound labeled antibody, the tissue sample is read for the presence of labeled immune complexes.

When used in vivo for therapy, the immunotoxins are administered to the patient in therapeutically effective amounts (i.e., amounts that eliminate or reduce the patient's tumor burden). They will normally be administered parenterally, preferably intravenously. The dose and dosage regimen will depend upon the nature of the cancer (primary or metastatic) and its population, the characteristics of the particular immunotoxin, e.g., its therapeutic index, the patient, and the patient's history. The amount of immunotoxin administered will typically be in the range of about 0.1 to about 10 mg/kg of patient weight.

For parenteral administration the immunotoxins will be formulated in a unit dosage injectable form (solution, suspension, emulsion) in association with a pharmaceutically acceptable parenteral vehicle. Such vehicles are inherently nontoxic and nontherapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives. The immunotoxin will typically be formulated in such vehicles at concentrations of about 0.1 mg/ml to 10 mg/ml.

The following examples are given for the purpose of illustrating various embodiments of the invention and are not meant to limit the present invention in any fashion.

### EXAMPLE 1

Purification of Gelonin

Seeds of Gelonium multiflorum were shelled and the nuts ground in a homogenizer with eight volumes of 0.14 M NaCl containing 5 mM sodium phosphate (pH 7.4). The homogenate was left overnight at 4° C. with continuous stirring, cooled on ice and centrifuged at 35,000 times g for 20 minutes at 0° C. The supernatant was removed, dialyzed against 5 mM sodium phosphate (pH 6.5) and concentrated using a pm10 filter. The sample was layered on a CM-52 ion-exchange column (20×1.5 cm) equilibrated with 5 mM sodium phosphate (pH 6.5). Material which bound to the ion exchange resin was eluted with 400 ml of 0 to 0.3 M linear NaCl gradient at a rate of 25 ml hour at 4° C. Five ml fractions were collected. The fractions were monitored at 280 nm in a spectrophotometer. The gelonin eluted in about fractions 55-70 and was the last major elution peak. Fractions 55-70 were pooled, dialyzed against double distilled water and concentrated by lyophilization. The purity and the molecular weight of each preparation was checked on high pressure liquid chromatography using a TSK 3000 gel permeation column with 50 mM sodium phosphate buffer, pH 7.4 and 15% sodium dodecylsulphate-polyacrylamide gel electrophoresis (SDS-page). Gelonin migrated as a single band with an approximate molecular weight of 29-30,000 daltons.

### EXAMPLE 2

Assay of Gelonin Activity

The gelonin activity was monitored in a cell-free protein synthesis inhibition assay. The cell-free protein synthesis inhibition assay was performed by sequentially adding to 50 μl rabbit reticulocyte lysate, thawed immediately before use, mixing after each addition, the following components: 0.5 ml of 0.2 M Tris HCl (pH 7.8), 8.9 ml of ethylene glycol, and 0.25 ml of 1 M HCl).

Twenty microliters of a salt-amino acid-energy mixture (SAEM) consisting of: 0.375 M KCl, 10 mM Mg(CH₃CO₂)₂, 15 mM glucose, 0.25-10 mM amino acids (excluding leucine), 5 mM ATP, 1 mM GTP, 50 mM Tris-HCl (pH 7.6), 10 μl Creatinine phosphate-creatinine phosphokinase, 8 μl ¹⁴C leucine (Amersham, 348 mCi mmol), and adding 1.5 μl of solutions containing varying concentrations of the gelonin mixture. The mixture was incubated for 60 minutes at 30° C. ¹⁴C-leucine incorporation was monitored in an aliquot of the mixture by precipitating synthesized protein on glass fiber filters, washing in 10% TCA and acetone, and monitoring the radioactivity in a Beta-counter using Aquasol scintillation fluid. Gelonin with a specific activity no lower than 4×109⁹ U/mg was used for conjugation with the antibodies. A unit of gelonin activity is the amount of gelonin protein which causes 50% inhibition of incorporation of [¹⁴C] leucine into protein in the cell free assay.

### EXAMPLE 3

Modification of Gelonin with Iminothiolane

Gelonin in phosphate buffered saline was concentrated to approximately 2 milligrams/ml in a Centricon 10 microconcentrator. Triethanolamine hydrochloride (TEA/HCl), pH 8.0 and EDTA were added to a final concentration of 60 mM TEA/HCl and 1 mM EDTA pH 8.0. 2-Iminothiolane stock solution (20 mM) was added to a final concentration of 1 mM and the sample was incubated for 90 minutes at 4° C. under a stream of nitrogen gas.

Excess iminothiolane was removed by gel filtration on a column of Sephadex G-25 (1×24 cm) pre-equilibrated with 5 mM bis-tris acetate buffer, pH 5.8 containing 50 mM NaCl and 1 mM EDTA. Fractions were analyzed for protein content in microtiter plates using the Bradford dye binding assay. Briefly, forty microliters of sample, 100 μl of phosphate buffered saline (PBS) and 40 μl of dye concentrate were added to each well. Absorbance at 600 nm was read on a Dynatech Microelisa Autoreader. Gelonin elutes at the void volume (about fractions 14-20). These fractions are pooled and concentrated by use of a Centricon-10 microconcentrator.

### EXAMPLE 4

Monoclonal Antibody to ZME Antigen and Antibody-Secreting Hybridomas

One 8-week-old female BALB/c St mouse (SCRF Breeding Colony, La Jolla, Calif.) was injected intraperitoneally (i.p.) with 10⁷ human melanoma M21 cells and boosted i.p. with 5×10⁶ M21 cells 2 weeks later. One 50-week-old male NZB/B mouse (SCRF Breeding Colony) was primed with 5×10⁶ BW5 melanoma cells and boosted with 5 injections of 5×10⁶ BW5, M51, Colo 38, BW5 and M21 melanoma cells at monthly intervals. Three days after the booster injection, the mouse was sacrificed, and the spleen was removed, splenocytes were dissociated with a scalpel to make a cell suspension. The spleen cell suspension was treated with 0.17 ml NH₄Cl in 0.01 M Tris, pH 7.2, for 10 minutes to lyse the red blood cells. Then, these splenocytes were fused with SP2/0Ag14 cells as described by Gefter et al. ((1977) Somat. Cell Genet. 3:231.) with the following minor modifications: 5×10⁷ spleen cells and 10⁷ SP2/0Ag14 cells were hybridized with 0.3 ml of 30% (v/v) polyethylene glycol 1000 (PEG) in MEM. After incubation with PEG the cells were washed, and cultured at a concentration of 2×10⁶ cells/ml in D-MEM overnight. The next day, the cells were suspended in 40 ml HAT medium and pipetted into about 400 wells (0.1 ml/well) of microtiter plates. One drop (approximately 25 ml) of HAT medium was added at weekly intervals. After 2-3 weeks, the hybridomas selected for further studies were cultured in D-MEM with 10% FBS. Hybridomas were expanded in tissue culture and were grown in the peritoneal cavity of BALB/c mice primed with 0.5 ml of Pristane (Pfaltz and Bauer, Inc., Stamford, Conn.). The spent culture medium and ascitic fluid were used as source of antibody.

Clones of the hybridoma were grown in vitro according to known tissue culture techniques such as is described by Cotten, et al., Eur. J. Immunol. 3: 136 (1973). Hybridomas producing antibodies which reacted with human melanoma cells but not with normal human cells were further characterized. As shown on TABLE I, the antibodies produced by the ZME cell line and hybridomas-producing functionally equivalent antibodies reacted with the ZME antigen on human melanoma cells such as M-21.

TABLE I
NORMAL TISSUE REACTIVITY OF
ANTIBODY ZME-018 (225.285)*

| TISSUE | REACTIVITY** |
|---|---|
| Bladder | 0/3 |
| Brain Cortex | 0/2 |
| Cartilage | 0/2 |
| Colon | 0/2 |
| Nipples | 1/2 |
| Kidney | 0/2 |
| Lung | 0/4 |
| Lymph Node | 0/3 |
| Mammary Gland | 0/3 |
| Peripheral Blood Lymphocytics | 0/4 |
| Prostate | 0/2 |
| Salivary Gland | 0/3 |
| Skin | 0/5 |
| Stomach | 0/3 |
| Thyroid | 0/2 |
| Tonsil | 0/1 |
| Testes | 0/5 |

*From P. Giacomini, et al. Cancer Research 44: 1281-1287, 1984
**Number of samples antigen positive/number samples tested.

### EXAMPLE 5

Modification of Monoclonal Antibody ZME-018 with SPDP

N-succinimidyl 3-(2-pyridyldithio) (propionate)(SPDP) in dimethylformamide was prepared as a stock solution of 3 mg/ml in dry dimethylforamide. Since the crystalline SPDP can undergo hydrolysis, the actual concentration of chemically reactive crosslinker was determined by spectrophotometric methods by analyzing the absorbance at 260 nm in a dual-beam spectrophotometer. The concentration of SPDP stock is calculated from the following equation:

$$\frac{Change in absorbance ( 260 nm )}{0.02 \times 103 ml mmol} \times \frac{( 3.01 )}{0.01} = mmoles / ml / SPDP$$

One milligram of monoclonal antibody ZME in 1.0 ml of phosphate buffered saline (PBS) was added to a glass tube. SPDP stock solution was slowly added at about a 5-fold molar excess to the tube (approximately 10 μl of stock solution), mixing constantly. The mixture was incubated for 30 minutes at room temperature, mixing every 5 minutes during the incubation period.

Excess unreacted SPDP was removed from the sample by gel filtration chromatography on a Sephdex G-25 column (1×24 cm) pre-equilibrated with 100 mM sodium phosphate buffer pH 7.0 containing 0.5 mM EDTA (Buffer A). Fractions (0.5 ml) were collected and analyzed for protein content using the Bradford dye binding assay. Absorbance (600 nm) was monitored in a 96-well plate using a Bio-TEK Microplate autoreader. Antibody eluted at the void volume (fractions 14-20) and these fractions were pooled and kept at 4° C. The protein was concentrated in a Centricon-30 microcentrator. The Centricon retentate was washed with 100 mM sodium phosphate buffer, pH 7.0 containing EDTA (0.5 mM). The antibody was concentrated to a final volume of approximately 0.5-0.75 ml.

### EXAMPLE 6

Conjugation of SPDP-Modified Monoclonal Antibody ZME-018 with Iminothiolane-Modified Gelonin

One milligram of purified gelonin (2 mg/ml in PBS) prepared as described in Example 1 was modified with iminothiolane as described above. Monoclonal antibody ZME modified as described was mixed with an equal weight of gelonin modified as in Example 3. This proportion corresponded to a 5-fold molar excess of gelonin as compared to antibody. The pH of the mixture was adjusted to 7.0 by the addition of 0.05 M TEA/HCl buffer pH 8.0 and the mixture was incubated for 20 hours at 4° C. under nitrogen. Iodoacetamide (0.1 M) was added to a final concentration of 2 mM to block any remaining free sulfhydryl groups and incubation was continued for an additional hour at about 25° C. The reaction mixture was stored at 4° C. until purification by gel filtration.

### EXAMPLE 7

Purification of Gelonin-Monoclonal Antibody 15A8 Complexes

Non-conjugated gelonin and low molecular weight products were removed from the reaction mixtures of Example 6 by gel filtration on a Sephadex S-300 column (1.6×31 cm) pre-equilibrated with PBS. Reaction mixtures from Example 6 were concentrated to approximately 1 ml with a before loading on the Sephadex column. The column was washed with PBS. One ml fractions were collected and 50 μl aliquots are analyzed for protein by the Bradford dye binding assay. As shown on FIG. 2, free- and gelonin-conjugated antibody eluted in the void volume (about fractions 28-40) while, unconjugated gelonin elutes at about fractions 45-65.

To remove unconjugated ZME-018, the high molecular peak (fraction 28-40) from the S-300 column was applied to an affinity chromatography column of Blue Sepharose CL-6B (1×24 cm) pre-equilibrated with 10 mM phosphate buffer (pH 7.2) containing 0.1 M NaCl. After sample loading, the column was washed with 30 ml of buffer to completely elute non-conjugated antibody. The column was eluted with a linear salt gradient of 0.1 to 2 M NaCl in 10 mM phosphate buffer pH 7.2. Protein content of the eluted fractions was determined.

FIG. 2 shows the elution profile of the S-300 column and demonstrates that gelonin was separated from gelonin-antibody conjugate and unconjugated antibody, both of which co-elute in the first peak (about fractions 28-40). This elution pattern was confirmed by electrophoresis of 50 μl aliquots on 5-20% gradient non-reducing SDS polyacrylamide gels as shown on FIG. 4. The coupling mixture was loaded on lane 3. Bands for free gelonin (lane 2), free antibody (lane 1) and for one molecule of gelonin coupled per molecule of antibody and two molecules of gelonin coupled per antibody molecule are shown. The void volume peak of the S-300 column containing free antibody and antibody-gelonin conjugate was loaded on lane 4. Non-conjugated antibody was removed from the gelonin conjugated antibody by affinity chromatography on a column (1×24 cm) of Blue Sepharose CL-6B pre-equilibrated with 10 mM phosphate buffer, pH 7.2 containing 0.1 M NaCl. After loading the S-300 eluate sample, the column was washed with 30 ml of the same buffer to completely elute non-conjugated antibody.

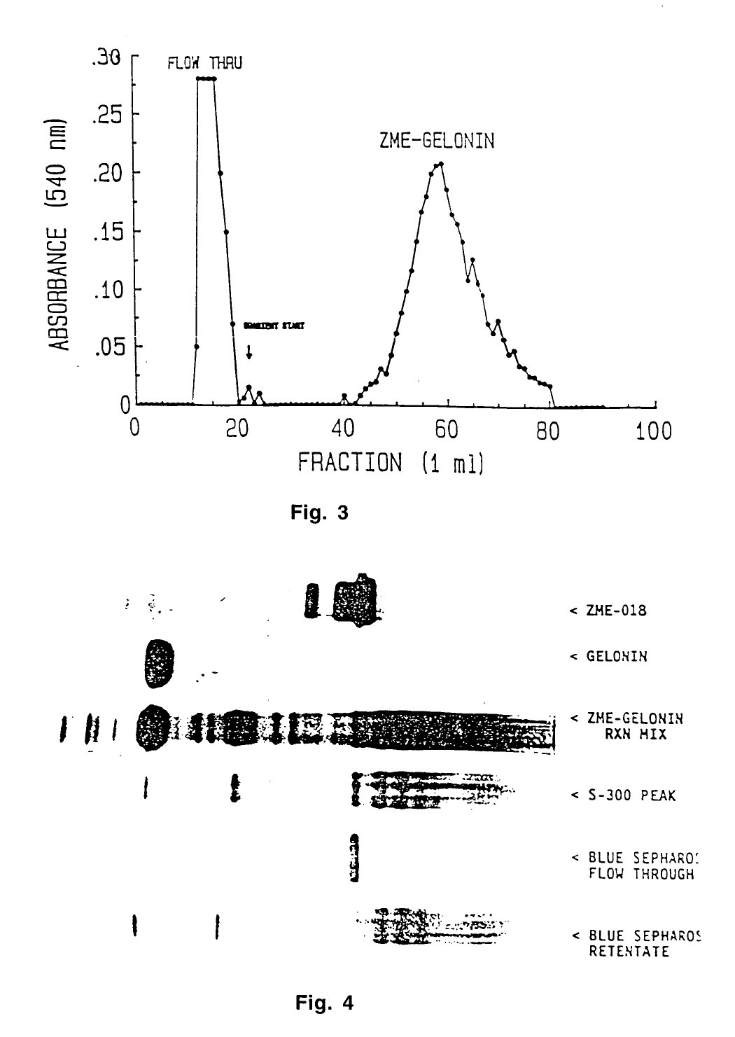

Gelonin-conjugated antibody bound to the column and was eluted with a linear salt gradient of 0.1 to 2 M NaCl in 10 mM phosphate buffer, pH 7.2. The antibody-gelonin complex eluted at approximately 0.7 M NaCl as shown on FIG. 3 which depicts the elution profile of the Blue Sepharose column. Protein content of the eluted fractions was determined. The protein-containing fractions were pooled and the elution pattern confirmed by electrophoresis on a 5 to 20% gradient non-reducing polyacrylamide gel. The electrophoretic pattern of the ZME-gelonin complex is shown on FIG. 4. The flow-through peak (fractions 14-20) contains only free antibody (FIG. 4, lane 5) while fractions 50-80, eluted with high salt, contain ZME-gelonin conjugate free of unconjugated gelonin or antibody (FIG. 4, lane 6). The final product contained ZME antibody coupled to 1, 2 and 3 gelonin molecules. Average gelonin content was 1.5 molecules per antibody molecule.

The rabbit reticulocyte in vitro translation system was utilized to estimate the gelonin activity of the essentially pure gelonin-ZME antibody complex. One unit of activity in this assay was defined as the amount of protein required to provide 50% inhibition of protein synthesis as compared to untreated controls. Utilizing this assay, the specific activity of both the native gelonin and the ZME-gelonin conjugate were determined to be 2×10⁸ U/mg and 8.2×10⁵ U/mg respectively. The essentially pure gelonin-ZME antibody was active in the reticulocyte lysate assay. A 1:1000 dilution of the original sample caused approximately a 50% inhibition of protein synthesis, i.e., a 50% reduction of the incorporation of ¹⁴C-leucine into protein. The activity of the original preparation was 1000 U/ml.

### EXAMPLE 8

Cell Culture Methods

ZME antigen-negative human bladder carcinoma (T-24) human cervical carcinoma or ZME antigen-positive human metastatic melanoma tumor cells A375M or AAB-527 were maintained in culture using minimal essential medium supplemented (MEM) with 10% heat-inactivated fetal bovine serum plus 100 mM non-essential amino-acids, 2 mM L-glutamine, 1 mM sodium pyruvate, vitamins and antibiotics. Cultured cells were routinely screened and found free of mycoplasma infection.

Cell Proliferation Assay

Cell lines were maintained in culture in complete medium at 37° C. in a 5% CO₂-humidified air incubator. For assays with combinations of TNF, immunotoxins, IFNγ, and IFNα, cultures were washed, detached using versene, and resuspended in complete medium at a density of 25×10³ cells/ml. Two hundred ml aliquots were dispensed into 96-well microtiter plates and the cells were then allowed to adhere. This results in a sparsely seeded population of cells. After 24 hours the media were replaced with media containing different concentration of either immunotoxins, toxins, TNF, IFNα, or IFNγ. The cells were incubated for 72 hours and analyzed for relative cell proliferation by crystal violet staining.

Crystal Violet Staining

Cells were washed 3 times with PBS containing calcium and magnesium fixed and stained with 20% (v/v) methanol containing 0.5% (v/v) crystal violet. Bound dye was eluted with 150 μl of Sorenson's citrate buffer (0.1M sodium citrate, pH 4.2-50% (v/v ethanol) for 1 hour at room temperature. The absorbance was measured at 600 nm using a Bio-Teck microplate reader. Relative cell proliferation (RCP) was calculated as follows:

$$RCP = \frac{Mean Absorbance ( Drug Treated )}{Mean Absorbance ( Non - drug Treated )} \times 100$$

Human Tumor Colony Assay

Tumor biopsy specimens were obtained from melanoma patients during clinically indicated biopsy procedures. Tumor cell suspensions were prepared aseptically (Leibovitz, et al., Int. J. Cell Cloning 1: 478-485 (1983)). Additionally, the A375P melanoma and the CEM leukemia cell lines from the American Type Culture collection were also tested. Testing the effects of ZME-gelonin on the fresh melanoma cell suspensions and cell lines was assessed in HTCA using standardized procedures for tumor cell plating in semisolid medium (agarose) in the presence of complete medium containing 10% fetal calf serum, each 0.5 ml culture plate containing 100,000 cells for fresh tumors and 10,000 cells for the cell lines. ZME-gelonin was tested by addition to the culture plates shortly after tumor cell plating. ZME-gelonin was added to triplicate plates at each of four concentrations 0.025 ng/ml to 250 ng/ml. In addition to untreated control plates, unconjugated ZME-18 monoclonal antibody and free gelonin were tested in parallel. Cell lines and tumor cell cultures were incubated for an average of 10 days at 37° C. in 5% CO₂ in air in a humidified incubator, and colony formation evaluation with a viability stain and an automated image analysis instrument optimized for colony counting. Percent survival of ZME-018 treated cultures in relation to simultaneous untreated controls were determined in the same experiments. Dose-response curves were then plotted graphically.

### EXAMPLE 9

Binding of Gelonin-Conjugated and Unconjugated ZME Antibody

The ability of the gelonin-conjugated and unconjugated ZME antibody to bind to target cells was assessed. The binding of ZME-gelonin immunotoxin to antigen positive (AAB-527 cells) or antigen negative (T-24 cells) was tested by ELISA assay. Fifty thousand target cells (AAB-527 cells) or non-target (T-24 cells) were added to each well of microtiter plate. The cells were dried on the plates overnight at 37° C. The cells were then washed with three changes of cold PBS and air dried overnight. The cell surface antigenic determinants remain antigenically active.

After attachment of the cells, the plates were washed with Washing Buffer (9.68 g Tris, 64.8 g sodium chloride, 16 ml Tween 20, 800 mg thimerasol in 8 l of double distilled water). Antibody samples were diluted in Washing Buffer containing 1% bovine serum albumin (w/v) (Diluting buffer). Fifty microliters of various concentrations ranging from .02 to 50 μg/ml of either conjugated or unconjugated ZME antibody were added to the wells. After incubation for 1 hour at 4° C., the supernantants were removed and the wells washed twice with Washing Buffer.

Fifty microliters per well of horseradish peroxidase conjugated goat anti-mouse IgG obtained from Bio-Rad and diluted 1:1000 (v/v) (HPGAM) in Diluting buffer was added to each well. The plates were incubated for 1 hour at 4° C. and the wells washed twice with Washing Buffer. After incubation of the plates with 50 ul of Substrate Solution (80 mM citrate phosphate (pH 5.0), 1 mM 2,2′ Azino-Bis (3-Ethyl Benz-Thiazoline-6-Sulfonic Acid) (Abts) Diammonium Salt (Sigma Chemical Co) and 4 μl of 30% hydrogen peroxide) in the dark for 30 minutes at room temperature, 25 μl of 4 N sulfuric acid was added to each well. The absorbance at 492 nm was determined.

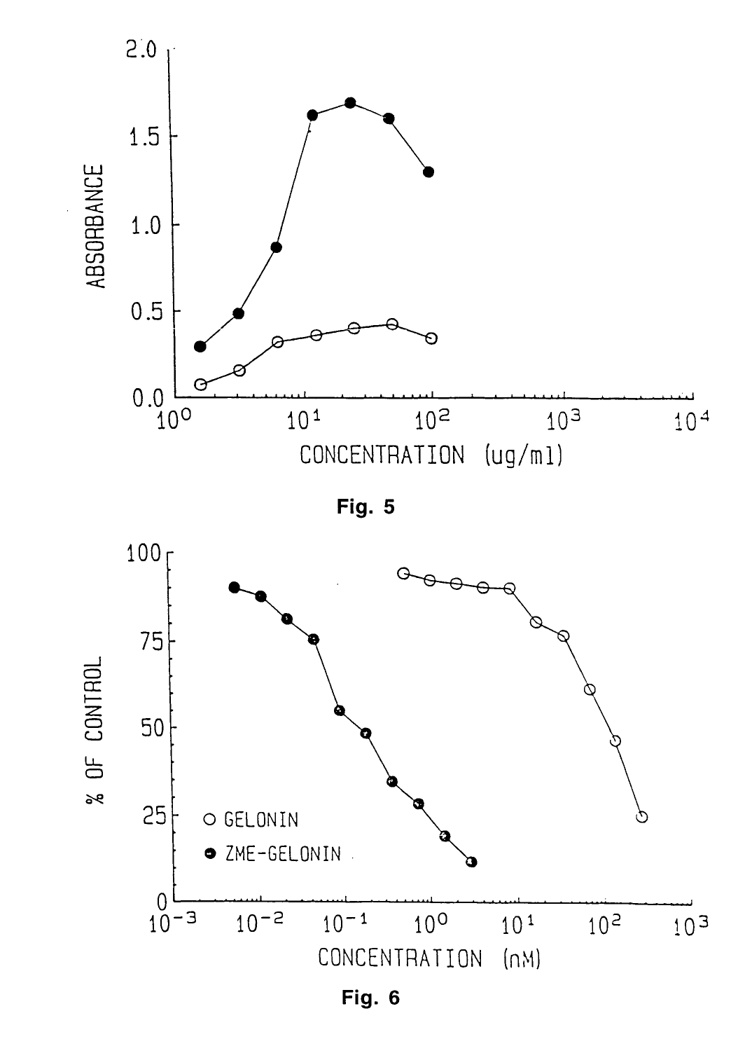

As shown in FIG. 5, both native ZME and the ZME gelonin conjugate bound well to target cells after 60 minute exposure. Surprisingly, the ZME-gelonin conjugate bound target cells better than did the native antibody. This increase was not due to modification of the antibody by SPDP since SPDP-modified ZME behaved identically to that of native ZME. The increase was also not due to binding of target cells to the gelonin portion of the molecule since pre-treatment of target cells with native gelonin had no effect on either antibody or immunotoxin binding. Neither ZME nor ZME-gelonin bound to antigen negative T-24 cells as estimated by ELISA assay.

### EXAMPLE 10

Cytotoxicity of Gelonin and Gelonin-ZME Antibody Complex

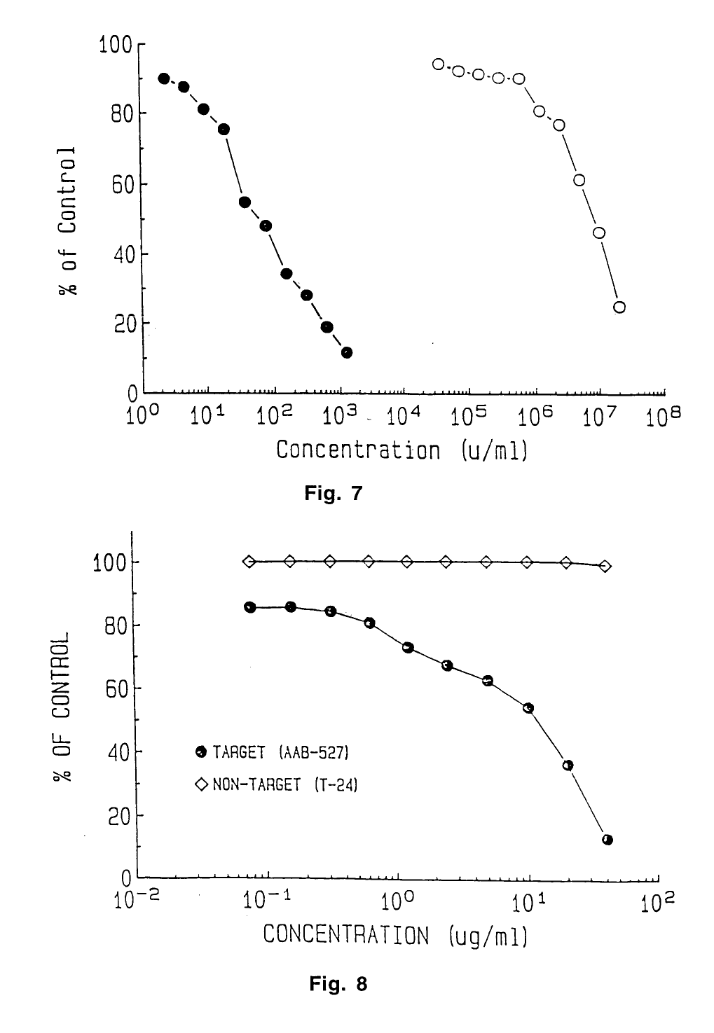

Cytotoxicity studies of the ZME-gelonin conjugate were performed on antigen-positive cells after continuous (72 hour) exposure to the immunotoxin or native gelonin. As shown in FIG. 6, when antigen-positive AAB-527 cells were exposed to approximately 0.1/nM ZME gelonin, 50% cell death was observed. When cells were exposed to native gelonin, a concentration of 100 nM gelonin was required to reduce the cell number to 50% of the untreated control. Target cells were then treated with various concentrations of ZME-gelonin or gelonin alone on a unit basis. As shown in FIG. 7, 50% cytotoxicty was obtained using 50 units/ml of ZME-gelonin conjugate while 1×10⁷ units/ml of the free gelonin were required to achieve the same effect.

The effect of ZME-gelonin was determined against antigen-negative T-24 cells in log-phase culture. As shown in FIG. 8, gelonin alone produced 50% cytotoxicity in AAB-S27 cells at a concentration of 100 μg/ml, similar to that found on AAB-S27 cells. ZME-gelonin produced 50% cytotoxicity in target T-24 cells at a concentration of 10 μg/ml. However, the ZME-gelonin immunotoxin was not cytotoxic against non-target T-24 cells even at the highest concentration tested.

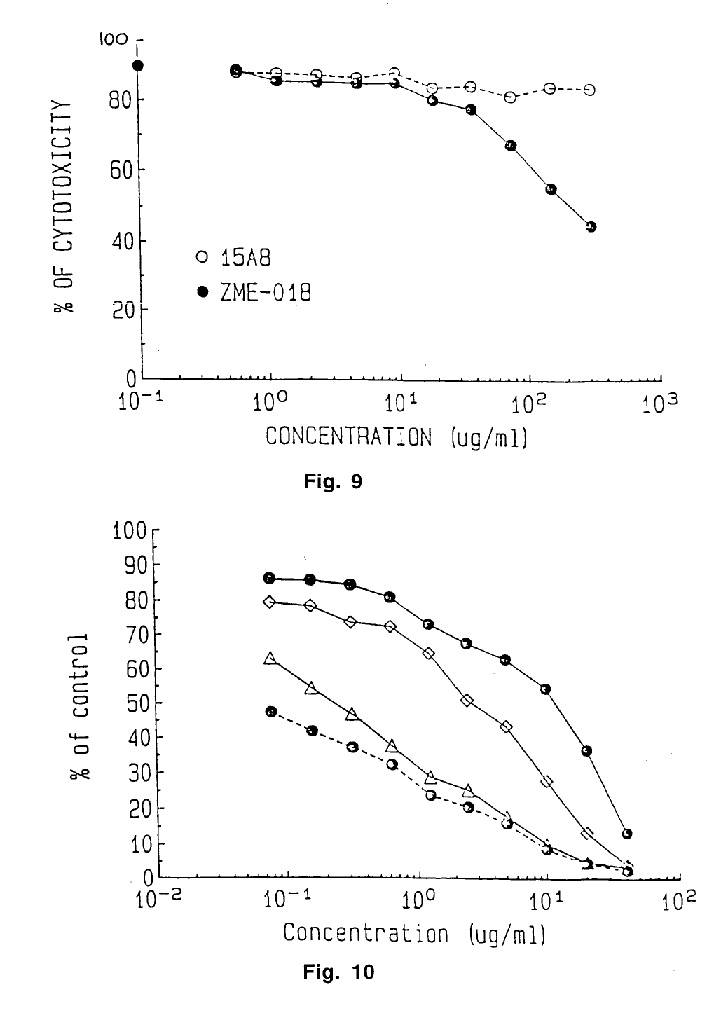

In order to further demonstrate that the ZME gelonin cytotoxicity was mediated through the ZME cell surface antigen, a fixed dose of ZME-gelonin which achieves 80% cytotoxicity was added to target log phase melanoma cells in culture in the presence of free ZME antibody or an irrelevant antibody (15A8, an antibody that does not bind to melanoma cells). As shown on FIG. 9, the presence of increasing amounts of ZME antibody suppressed the cytotoxicity of the ZME-gelonin conjugate while the 15A8 antibody had no effect. Thus, the cytotoxicity of the ZME-gelonin conjugate was directly mediated by the binding of the ZME antibody to ZME antigen on the target cell.

### EXAMPLE 11

Modulation of ZME-Gelonin Cytotoxicity with IFNγ, IFNμ and TNF.

To demonstrate the effects of treatment with various biological response modifiers on immunotoxin cytotoxicity, log-phase melanoma cells were treated for 24 hours with fixed doses of IFNα (200 u/ml), IFNγ(20,000 u/ml) or rTNF-α (20,000 u/ml). These doses were previously determined to have minimal effect (approximately 20%) cytotoxic effect against these cells. The cells were then treated for 72 hours with various doses of ZME-gelonin. As shown in FIG. 10, treatment with rIFNγ resulted in a 2-fold increase in sensitivity to the ZME-gelonin immunotoxin. However, pre-treatment with both rIFNα and TNF both resulted in a 2-log increase in sensitivity to the immunotoxin. Addition of fixed doses of rIFNα, rIFNγ or rTNF to antigen-positive cells resulted in augmented cytotoxicity of the ZME-gelonin toxin. Treatment with rTNF-α caused the greatest increase in immunotoxin cytotoxicity followed by rIFNα and rIFNγ.

Substantial augmentation of ZME-gelonin cytotoxic effect was observed with pre-treatment of rIFNα and rTNF but not with rIFNγ. While it has been observed that IFNα and IFN-γ can up-regulate some melanoma surface antigens such as P-97, there was little effect of the agent on the high molecular weight antigen (GP 240) recognized by ZME. Therefore, the mechanism of TNFα and IFNα induced augmentation of ZME-gelonin activity is not clear but could involve changes in the antibody internalization rate, changes in the cellular processing of the immunotoxin or a modulation of any one of several interferon-mediated enzymes.

Since only cells containing the ZME antigen on their surface were killed by the gelonin ZME immunotoxin, this immunotoxin is an efficient method to target and kill ZME tumor associated antigen containing cells while minimizing or preventing damage or injury to normal non-tumor associated antigen-bearing cells.

### EXAMPLE 12

Effect of ZME-Gelonin Immunotoxin in Human Tumor Colony Assay (HTCA)

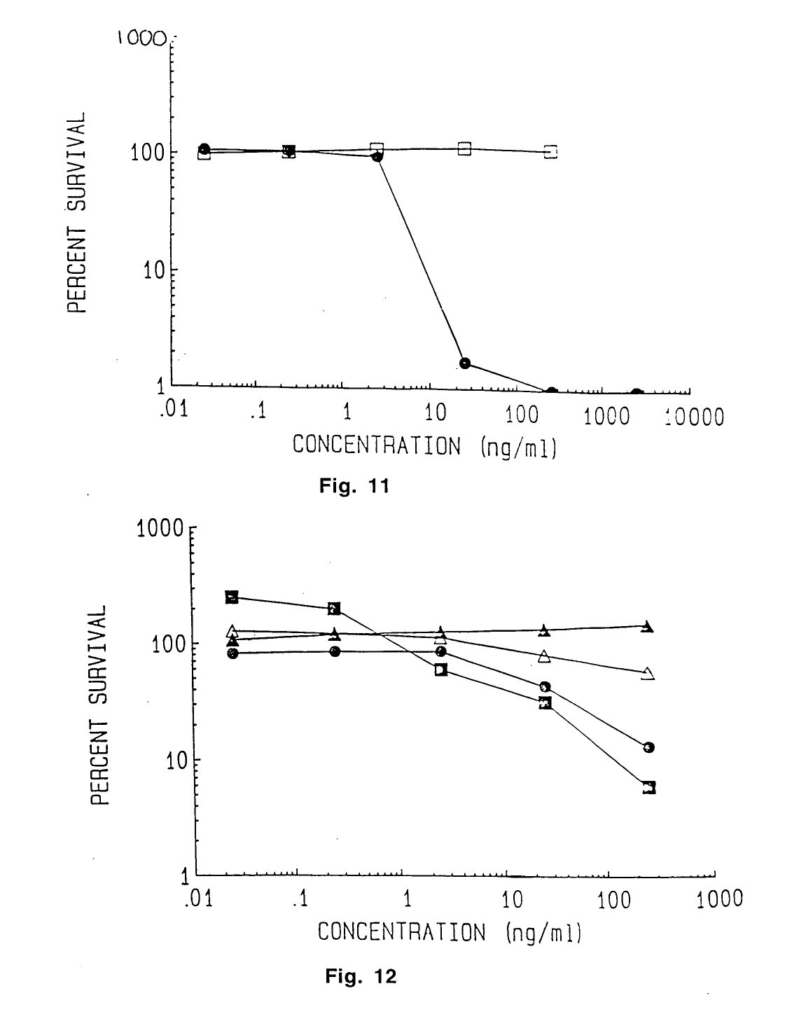

The activity of ZME-gelonin was also assessed using the human tumor colony assay against cells obtained from biopsy of four patients with melanoma. In vitro cytotoxicity against human cells in culture was also assessed in the HTCA described in Example 8C. Various doses of ZME-gelonin immunotoxin were added to an antigen positive (A-375 melanoma) and antigen negative CEM cell lines. Survival of colonies was assessed 72 hours after addition. As shown in FIG. 11, doses of immunotoxin between 0.25 and 2500 ng/ml resulted in almost complete supression of colony survival of the antigen-positive cell line (closed circle). ZME-018 and free gelonin alone or combined together were not cytotoxic. There was no effect against the antigen-negative line (CEM) even at the highest concentration of immunotoxin tested (open squares).

The effect of ZME-gelonin against 4 different fresh biopsy specimens is shown in FIG. 12. Eighty to 90% reduction in survival of melanoma colony forming cells was found in two specimens at the highest immunotoxin dose tested (250 ng ml). One patient showed 50% inhibition of cell growth at this dose, while one patient showed no cytotoxicity of the immuno-conjugate. A modest (25%) reduction in colony number was noted with a third specimen. Growth enhancement was noted in the fourth sample at the highest immunotoxin dose. In addition, growth enhancement was observed in one specimen at low doses, while higher doses produced substantial cytotoxicity. As in the cell line experiments, addition of unconjugated ZME-018 and free gelonin were not cytotoxic.

While the HTSCA assay is not infallable, approximately 75% of clinically active antitumor agents are positive. Agents inactive in the HTSCA are inactive clinically. Therefore, activity of the ZME-gelonin conjugate in the HTSCA provides one with ordinary skill in this art to reasonably expect at least a 75% probability of clinical value.

### EXAMPLE 13

Tissue Distribution of ZME Antibody

The tissue distribution of ¹²⁵I labeled ZME antibody was compared to ZME-gelonin immunotoxin and a control immunotoxin (15A8-gelonin). Each antibody or antibody-conjugate was administered intravenously in the tail vein to 5 nude mice bearing human melanoma xenografts. Each animal received 10 mg of total protein labeled with 0.5 μCi of ¹²⁵I in a total volume of 100 μl of phosphate-buffered saline.

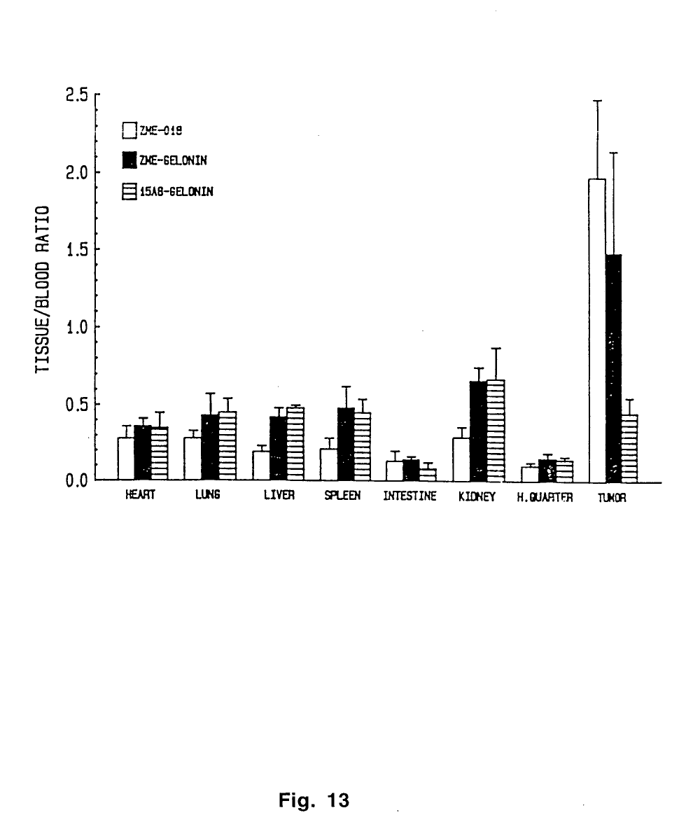

As shown in FIG. 13, the irrelevant 15A8-gelonin conjugate did not localize specifically in tumor tissues (T/B ration 0.5). In contrast, both the ZME and ZME-gelonin conjugate demonstrated specific localization (T/B ratios of 2.0 and 1.5 respectively). There was no statistically significant difference in the uptake of ¹²⁵I into tumor after ZME or ZME-gelonin administration.

### EXAMPLE 14

Cell Lines

The cultured human melanoma cell line A375-M was provided by Dr. I. J. Fidler of MD Anderson Cancer Center. The cells were grown in DMEM supplemented with 10% fetal calf serum (Hyclone Products, Logan, UT) 20 mM glutamine, 50 mg/ml gentamicin, non-essential amino acids and 100 mM sodium pyruvate (Cellgro Products). The cells were passaged twice a week, maintained at 37° C. and routinely tested for mycoplasma. The melanoma cell line AAB-527 was provided by Dr. B. Giovanella of the Stehlin Foundation, Houston, Tex. These cells were also maintained in DMEM containing 10% FCS and were routinely tested for mycoplasma. The coupling, purification and in vitro testing of Mab ZME-018 and gelonin are described in detail above. The immunoreactivity of the purified ZME-gelonin immunotoxin was examined by ELISA against antigen positive A375-M (melanoma) and antigen negative T-24 (bladder CA) cells. The in vitro efficacy of ZME-018 gelonin immunotoxin was also examined against antigen positive A375-M (melanoma) and antigen negative T-24 (bladder CA) cells using a 72 hour cytotoxicity assay described above.

### EXAMPLE 15

Antibody and Antibody-Toxin Labeling Using PIB

One drawback in the use of ¹²⁵I or ¹³¹I labeled protein in vivo is the potential for rapid and extensive dehalogenation. A procedure for radioiodination has been described and utilized for monoclonal antibodies which incorporates iodine into protein via a metabolically stable linkage Wilbur et al., J Nucl Med, 30:216 (1989). This method conjugates N-succinimidyl para-iodobenzoate to the Mab. Briefly, 37.5 ml of 1% HOAC/MeOH 10 ml of a 1 mg/ml solution of N-chlorosuccinimide (NCS) in MeOH and 10 ml of PBS were sequentially added to a reaction vial fitted with a rubber septum containing N-succinimidyl 4-tri n-butylstannylbenzoate (NeoRx Corp., Seattle, Wash.) (12.5 mg) in 12.5 ml of HOAC/MeOH. One mCi of ¹²⁵I (Dupont) was added to the reaction solution and after 5 minutes, the reaction was quenched by addition of 10 ml of 0.1 M NaHSO₃. The MeOH solvent was evaporated under a N₂ stream for 10 minutes. Five hundred mg of Mab in 100 ml PBS was mixed with 100 ml 0.5 M, of borate buffer (pH 9.3) and then added to the reaction vial. The conjugation was allowed to proceed for 5 minutes. at room temperature. Radiolabeled Mab was separated from unreacted radioiodine by chromatography on a Sephadex G-25 (PD-10) column (Pharmacia LKB Biotechnology, Piscataway, N.J.). Radiochemical yield was from 40-60%. Incorporation of radiolabel into Mabs measured by TCA precipitation was greater than 90%. The specific activity of radiolabeled Mabs ranged between 0.2-0.3 mCi/ug.

### EXAMPLE 16

Immunoreactivity Assay

The immunoreactivity of radiolabeled Mabs and immunotoxin was evaluated using the method described in Lindmo et al., J immuno Methods 72:77-79 (1984). Briefly, melanoma cells (2×10⁶ A375-M) were incubated with various concentrations of ¹²⁵I-labeled antibody or immunotoxin for 1 hour at 4° C. The cells were washed with PBS containing 1% BSA and lysed with 2% NP-40 (Sigma) counted in a gamma counter (Packard model 5360). The immunoreactivity values ranged from 40-60% for both ZME-gelonin immunotoxin and ZME-018 monoclonal antibody.

### EXAMPLE 17

Tissue Distribution

Four to six week old athymic (nu/nu) mice were obtained from Harlan Sprague Dawley, Indianapolis, Indiana. The animals were maintained under specific pathogen-free conditions and were used at 6-8 weeks of age. Animals were injected subcutaneously, (right flank) with 2×10⁶ log phase A375-M melanoma cells and tumors were allowed to establish for three weeks. Monoclonal antibodies and immunotoxins were labeled with ¹²⁵I 24 hours prior to injection at a specific activity 0.3 mci/ug protein. After examining the immunoreactivity of the antibody and immunotoxin, labeled ZME 018, ZME-gelonin immunotoxin and a control immunotoxin (15A8-gelonin directed against breast CA), mice were injected (i.v. tail vein) with 5 mCi of labeled antibody or immunotoxin in 100 ml of normal saline. Mice were sacrificed by cervical dislocation 24 and 72 hours following injection. Samples of tumor, heart, lung, liver, spleen, kidney, stomach, intestine and muscle were removed, weighed and assayed for radioactivity in a Packard gamma counter (model 5360). The percentage of injected Mab/g tissue (% ID/g) in tumor and normal organs was calculated. Tumor to blood or tumor to organ ratios were also calculated by dividing the % ID/g Mab in tumor by the % ID/g Mab in the respective organ.

### EXAMPLE 18

Pharmacokinetics

Four to six week old BALB/C mice were injected with 0.5 mCi (5 mg) (specific activity 4.65×10⁷ cpm/mg protein) of either labeled Mab ZME-018 or ZME-gelonin immunotoxin; at 10, 15, 30, 60, 90, 120, 180, 240 mins and 24 hrs after injection, 2 mice at each time-point were sacrificed by cervical dislocation. Blood samples were removed (chest cavity), weighed and counted to determine total radioactivity in a gamma counter (Packard, model 5360). The blood samples were also centrifuged and plasma was decanted and counted to determine radioactivity. Results from plasma determination of radioactivity were analyzed by a least-square nonlinear regression (RSTRIP, from MicroMath, Inc.) program to determine pharmacokinetic parameters.

### EXAMPLE 19

In Vivo Efficacy Study

4 to 6 week old BALB/C nude (nu/nu) mice were injected with 2×10⁶ A375-M log phase melanoma cells subcutaneously in the right flank. The tumors were allowed to establish for 3 weeks prior to starting therapy and the mice were divided into 4 groups. Each treatment group had 5 mice with 100-200 mm³ established tumors. The mice were injected (i.v. tail vein) with either PBS, gelonin (0.044 mg/injection/mouse), ZME-gelonin immunotoxin (0.22 mg/injection/mouse) twice per week for 3 weeks. At the end of three weeks of therapy, the mice were monitored for additional 30 days.

### EXAMPLE 20

Survival Using AAB-527 Human Melanoma Cells:

Thirty 6-8 week old female mice were injected i.p. with 1×10⁶ AAB-527 melanoma cells. Twenty-four hours after the initial tumor cell inoculum therapy was initiated. The mice were divided into 4 groups; each group containing 5 mice each and were injected i.p. (tail vein) either with PBS, gelonin (0.07 mg/injection/mouse), ZME (0.4 mg/injection/mouse) or ZME-gelonin conjugate (0.4 mg/injection/mouse) on days 1, 3 and 6 respectively. At the end of the therapy, the mice were monitored for an additional 120 days.

### EXAMPLE 21

In Vitro Effects of ZME-Gelonin

The ZME-gelonin immunotoxin was found to be immunoreactive and specific on only antigen positive melanoma cells as evaluated by ELISA. The immunotoxin was also found to be specifically cytotoxic against only target melanoma cells (A375; IC₅₀ 30 ng/ml) with no significant cytotoxicity exhibited on antigen negative T-24 bladder carcinoma cells even at immunotoxin doses as high as 500 ng/ml.

### EXAMPLE 22

Tissue Distribution

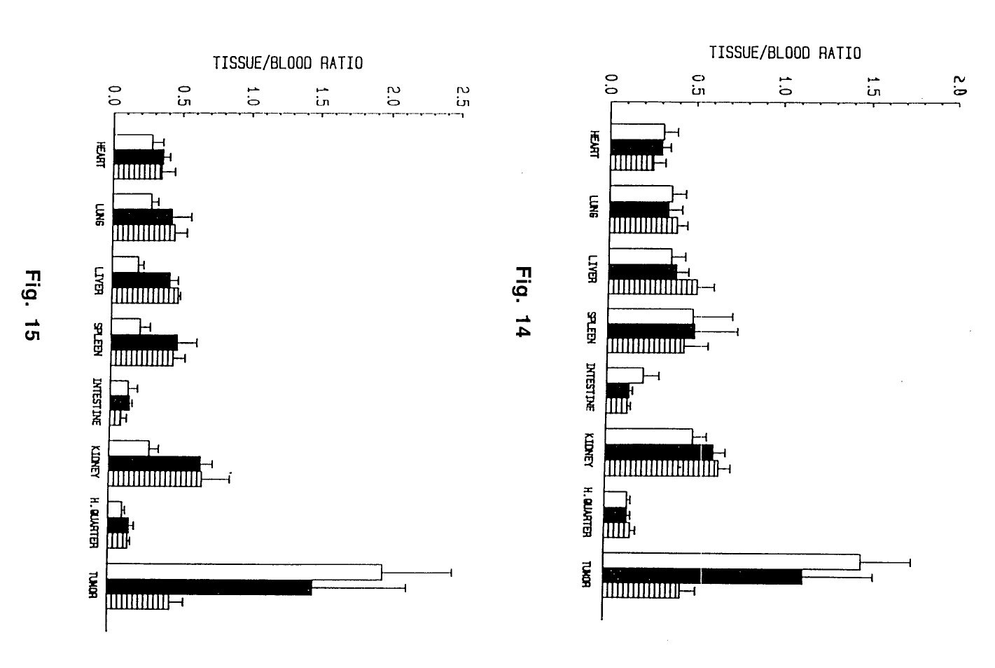

The tissue distribution of ¹²⁵I labeled ZME, ZME-gelonin or 15A8-gelonin (control immunotoxin) 24 or 72 hours after I.V. injection to groups of nude mice bearing well-established subcutaneous melanoma tumors is shown in FIGS. 14 and 15 respectively. At 24 hours after administration, (FIG. 14), both ZME and ZME-gelonin localized 2-3 fold greater in tumors compared to other normal organs. Tumor:blood ratio for ZME was 1.6±0.3 compared to 1.2±0.3 for ZME-gelonin. Tissue to blood ratios for all other organs ranged from 0.2 to 0.5. The highest uptake in normal organs was the kidney followed closely by spleen, liver and lung. The uptake of control immunotoxin closely paralleled that of ZME or ZME-gelonin in all normal organs. Uptake of control immunotoxins in tumor was similar to the uptake observed for spleen and liver.

At 72 hours after administration (FIG. 15), the content of ZME, ZME-gelonin and 15A8-gelonin were virtually identical in heart, lung, intestine and hindquarter. The content of ZME appeared slightly lower than the toxin conjugate (ZME-gelonin) in lung, liver, spleen and kidney. Uptake of irrelevant immunotoxin (15A8-gelonin) in tumor was similar to the content of the normal organs studied. The tumor content of ZME was 4-5 fold higher than normal organs (T:B 2.1±0.4) while the tumor content of ZME-gelonin was slightly lower (T:B 1.6±0.5).

### EXAMPLE 23

Plasma Pharmacokinetics of ZME and ZME-Gelonin

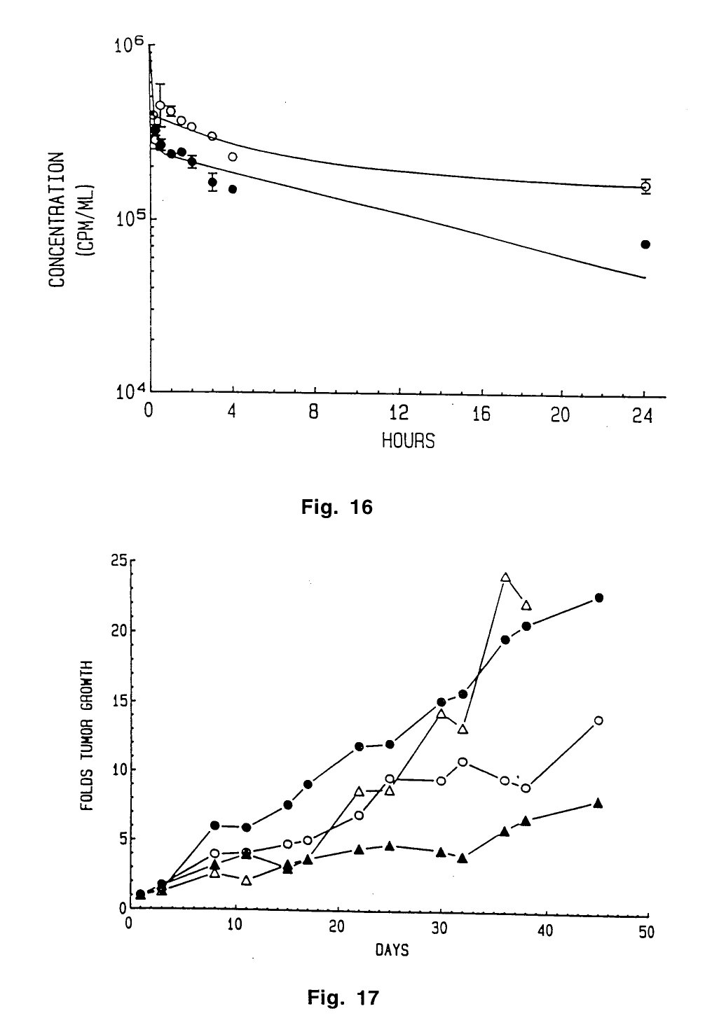

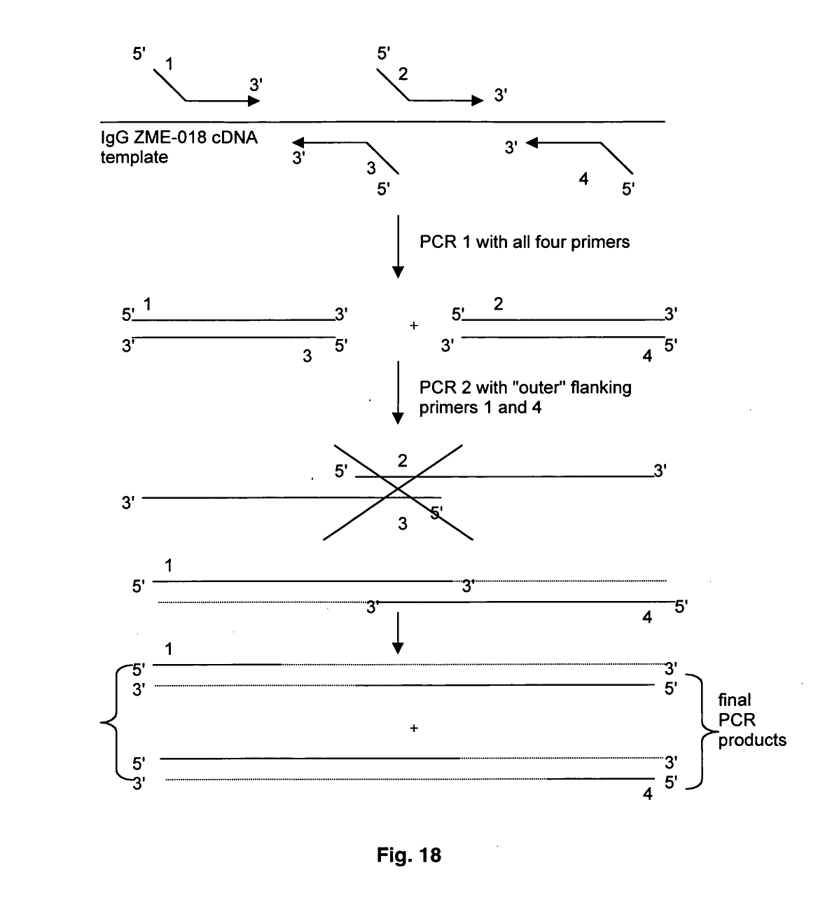

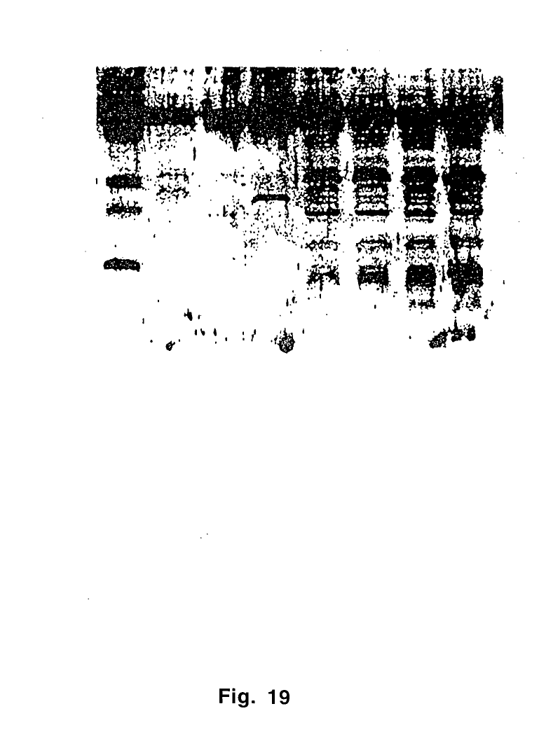

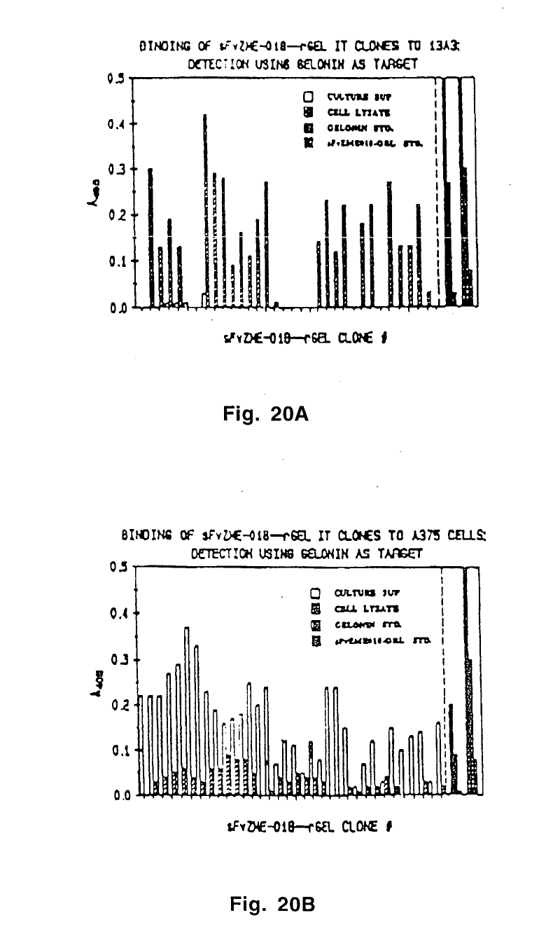

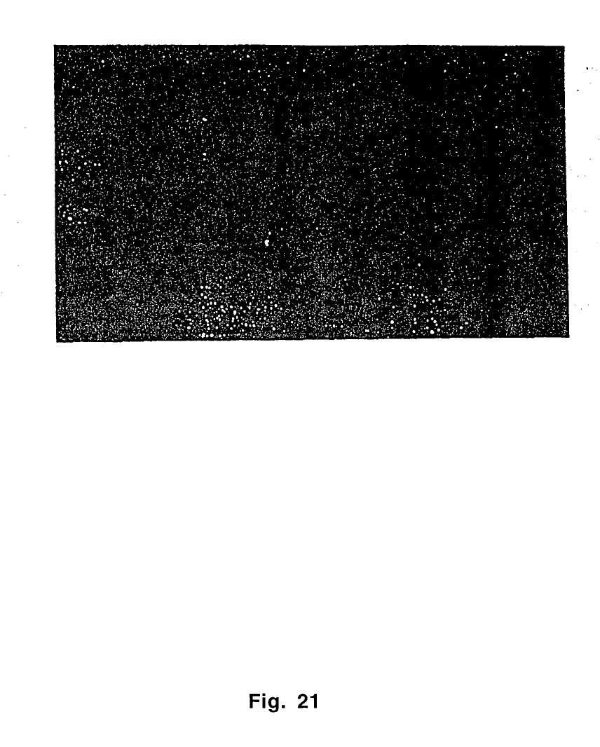

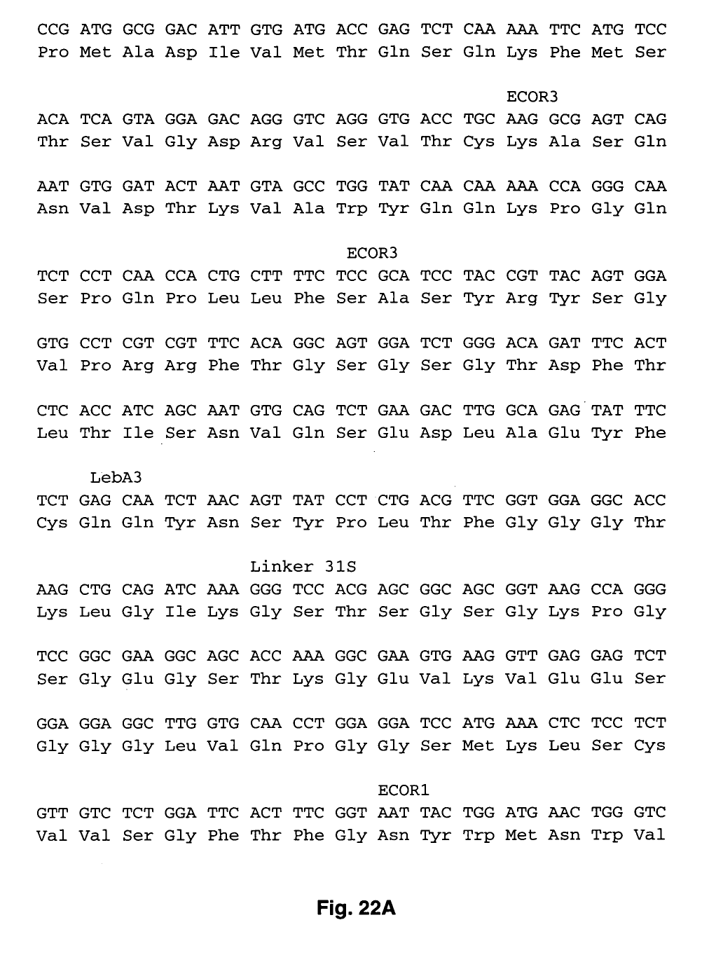

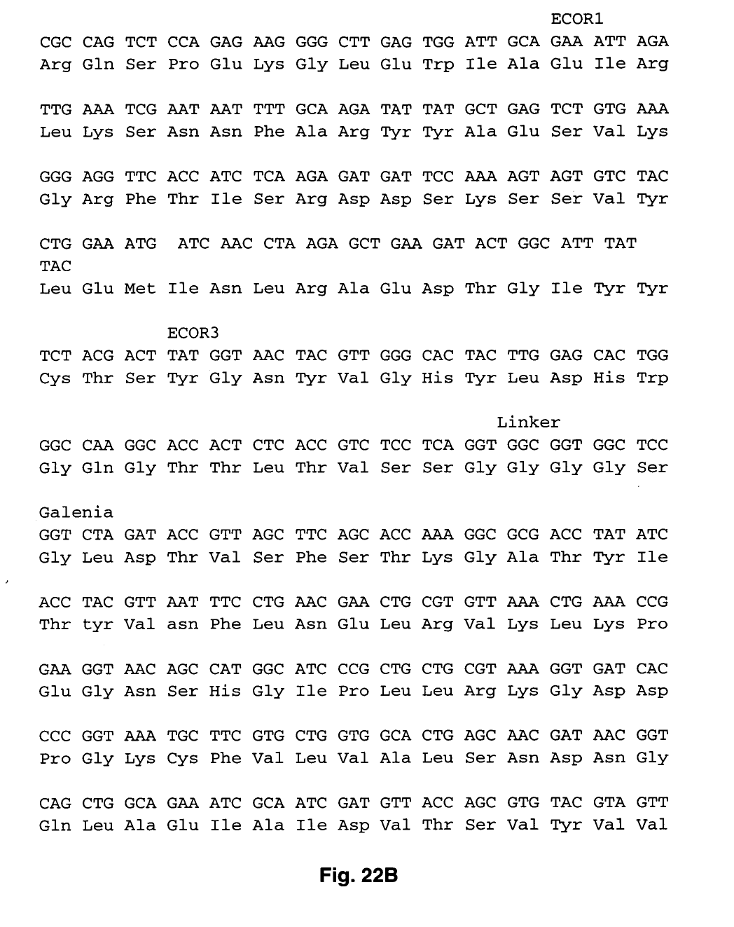

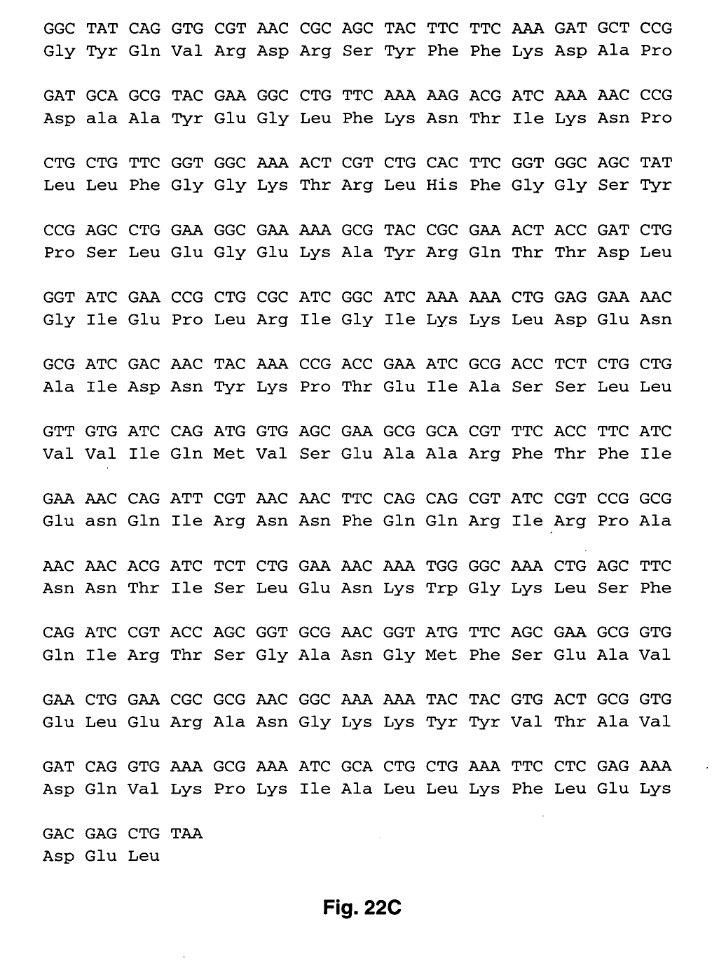

BALB/C mice were injected (I.V., tail vein) with 0.5 mci (5 mg) of radiolabeled ZME or ZME-gelonin conjugate. Blood samples were collected from mice at various times after injection. As shown in FIG. 16, the clearance of both ZME and ZME-gelonin conjugate fit a biphasic curve. Analysis of pharmacokinetic parameters (Table I) shows that the plasma half-life of the ZME-gelonin was shorter than ZME alone in both the α-phase (53.3 minute vs. 83.5 minute) and the β-phase (20.6 hours vs. 41.3 hours). The immediate apparent volume of distribution for the immunotoxin was 2.85 ml compared to 1.91 ml for ZME alone, suggesting more extensive distribution of the conjugate outside the vasculature. This is consistent with a lower Cxt for the conjugate and a higher clearance rate from plasma ((Clp) for the conjugate compared to that of native antibody.

TABLE 1
PHARMACOKINETICS OF ¹²⁵I-IMMUNOTOXINS
IN BALB-C MICE

| PARAMETER | ZME | ZME-GELONIN |
|---|---|---|
| Half Life (α)(min) | 83.50 | 53.30 |
| (β)(hrs) | 41.30 | 20.60 |
| Cp₀ (Ci/ml) | 0.24 | 0.16 |
| Vd (ml) | 1.91 | 2.85 |
| CXT (Ci/ml × min) | 139.60 | 80.80 |
| Clₚ (ml/kg. × min) | 0.16 | 0.28 |

### EXAMPLE 24

Antitumor Effects of ZME-Gelonin In Vivo

Nude mice bearing rapidly-progressing well-established human melanoma (A-375-M) tumors were treated (I.V.) with either saline, antibody, gelonin, or ZME-gelonin conjugate. Since pharmacokinetic and tissue disposition studies indicated that the tumor:blood ratio of the immunoconjugate was greater at 72 hour than at 24 hour after administration, the treatment schedule incorporated administration of therapeutic agents every 3 days. As shown in FIG. 17, tumor growth by day 45 in saline, gelonin and Mab ZME-018 treated controls increased 23, 22.2 and 14 fold respectively. In contrast, treatment of mice with ZME-gelonin conjugate demonstrated only a 8 fold increase in tumor volume. Compared to the gelonin and saline treated group, treatment with ZME-gelonin resulted in more than 50% suppression in tumor growth as measured by the increase in volume.

### EXAMPLE 25

Survival Study Using AAB-527 (Brown Tumor) Melanoma Cells

Nude mice were injected i.p. with 1×10⁶ AAB-527 melanoma cells. This is a rapidly growing metastatic model in which control group of mice die between 12-15 days after the initial tumor inoculum. The mice were injected with either saline, Mab ZME alone, gelonin alone (negative control), or ZME-gelonin immunotoxin. As shown in TABLE II, mean survival time of mice in PBS, ZME-018 and gelonin were from 14-16 days, whereas the mean survival time of mice treated with ZME-gelonin immunotoxin was ≈45 days. In addition, 1 mouse out of 5 survived up to 120 days. Thus, mice treated with ZME-gelonin is effective in prolonging the survival of nude mice bearing human melanoma cells, therefore demonstrating the in vivo efficacy of this immunotoxin.

TABLE II
Survival Of Balb/C/Nu:Nu Mice After
Melanoma (Aab-527) I.P. Injection

| Treated Group | Mean Survival (DAYS) | % Increase in MST |
|---|---|---|
| 1) PBS Saline | 14.2 + 2.2 | — |
| 2) ZME | 14.6 + 0.87 | 0 |
| 3) Gelonin | 16.2 + 4.71 | 14 |
| 4) ZME-Gelonin | 44.4 + 19.9 | 213 |

The present invention describes the construction and characterization of the in vitro activity of an immunotoxin composed of murine Mab ZME-018 and purified gelonin. The present invention also illustrates the highly potent and selective cytotoxicity of this conjugate against antigen-bearing cells. In t h e present invention demonstrates the pharmacology and in vivo therapeutic studies with purified ZME-gelonin conjugate. Tissue disposition studies of ZME-018 and ZME-gelonin radiolabels using the para-iodobenzoyl method revealed that the ZME-gelonin immunotoxin localizes in tumors as well as native antibody 72 hour after injection.

The plasma clearance of ZME-018 and ZME-gelonin conjugate both close fit a two-compartment mathematical model for clearance with half-lives for ZME-gelonin shorter than ZME for both the α-phase (53.3 min vs 83.5 min) and the β-phase (20 hrs vs 41.3 hrs).

The in vivo stability of RTA containing immunotoxins has been studied in a variety of models. Substantial degradation of the immunoconjugate has been noted leading to release of free RTA in plasma 24 hour after administration. In contrast, analysis by HPLC of plasma obtained from mice up to 24 hour after ¹²⁵I ZME-gelonin administration demonstrated no measurable release of free toxin. However, because of its relatively short half-life (≈3.5 minutes), free gelonin may not be observed under these conditions. Therefore, immunotoxins prepared with gelonin may show a greater inherent stability than immunotoxins prepared with other toxins such as RTA.

In the present invention, ZME-gelonin administration resulted in a 50% suppression of growth in a rapidly-growing melanoma model. The utility of ZME-gelonin was examined in an extremely aggressive melanoma model. In this model, death occurs due to metastatic tumors 14 days after the initial tumor inoculum. The immunotoxin treatment group demonstrated a 213% increase in MST compared to a saline-treated control group (14.2±2.2 days vs 44.4±19.9 days). In addition, in the ZME-gelonin treatment group, two animals survived >120 days after the initial tumor inoculum, thereby demonstrating the efficacy of ZME-gelonin immunotoxin in growth suppression of highly metastatic tumor foci present in diverse locations. In summary, ZME-gelonin conjugate demonstrates potent in vitro and in vivo antitumor effects against human melanoma.

### EXAMPLE 26

Single-chain antibodies (scFvs or sFvs), incorporating the binding characteristics of the parent immunoglobulin, consist of the antibody VL and VH domains (the Fv fragment) linked by a designed flexible peptide tether. The translation of scFvs as single polypeptides ensures expression of both VL and VH chains in equimolar concentrations and the covalent linking of the two sequences facilitates their association after folding. Compared to intact IgGs or Fab fragments, scFvs have the advantages of smaller size and structural simplicity with comparable antigen-binding affinities. In addition, they are more stable than the analogous two-chain Fv fragments. Furthermore, scFvs have significant advantages in clinical and diagnostic applications currently involving conventional monoclonal antibodies or Fab fragments thereof. The smaller size of scFvs provides for better penetration of tumor tissue, improved pharmacokinetics, and a reduction in the immunogenicity and high backgrounds observed with intravenously administered Fabs.

In another embodiment of the present invention, a single-chain analogue of the antibody ZME-018 was constructed which was raised against an epitope on the 240 kD antigen gp240 found on the surface of over 80% of melanoma cell lines and fresh tumor samples. The 18 amino acid 218 linker was chosen to tether the two variable regions of the antibody since this sequence was shown to provide for better proteolytic stability and degree of aggregation when compared to other heretofore commonly used linker peptides. Antibodies recognizing tumor cell-surface epitopes have the ability to selectively localize within human tumors after systemic administration and therefore have the potential to serve as targeting probes for the site-specific delivery of cytotoxic chemotherapeutic agents such as Pseudomonas exotoxin, ricin or gelonin. Therefore, an immunotoxin was constructed with sFvZME-018 and gelonin. In addition, with a view to increased efficacy of the immunotoxin, the carboxyl-terminal endoplasmic reticulum retrieval signal Lys-Asp-Glu-Leu (KDEL) was added to the sequence of gelonin. It is specifically contemplated that specific modifications in the sequence of scFvZME-018 such as CDR-grafting can be utilized to construct a humanized or chimeric antibody to minimize potential immunogenicity problems with the murine antibody.

### EXAMPLE 27

Construction of scFvZME-018

RNA was isolated from cells of FMT 112 P2, a murine hybridoma secreting the IgG2A antibody ZME-018 using standard methods. Messenger RNA was prepared using the Invitrogen Fast Track procedure and was immediately transcribed to cDNA with the Invitrogen Copy kit and oligo(dT) primers. Subsequently, the VL and VH antibody domains were amplified from the cDNA using the Novagen Ig-Prime protocol and cloned into the Invitrogen T/A cloning vector PCR II. Positive transformed clones were identified by blue-white screening and DNA from five clones for each of the two variable regions was sequenced using an Applied Biosystems 373A automated sequencer. The gene encoding scFvZME-018 was constructed, using a PCR-based method, in the VL-linker-VH configuration incorporating antibody DNA sequences identified from identical PCR II clones. Nco I and Spe I restriction sites were, respectively, engineered into the 5′ end of the LC DNA and 3′ end of the HC DNA via the primers sFvA (5′-GCTGCCCAACCAGCCATGGCGGACATTGTGATG) and sFVD (5′-GCCACCGCCACCACTAGTTGAGGAGACTGT-3′). The VL and VH DNA domains of the antibody were then linked together using the primers sFvB (5′-AAGCCAGGCTCCGGCGAAGGCAGCACCAAAGGCGAAGTGAAGTT-3′) a n d F v C 5 ′-GCCGGAGCCTGGCTTGCCGCTGCCGCTGGTGGAGCCTTTGATCACCAG. PCR syntheses were carried out in a Perkin Elmer Thermal Cycler and the profile used for the construction of the complete scFvZME-018 gene was as follows: the first step involved 25 cycles of 94° C. denaturation for 1.5 minutes, 50° C. annealing for 1.5 minutes, and a 1 minutes extension at 72° C. followed by a single 72° C. incubation for 5 minutes using all four aforementioned primers. Following this, one tenth volume of the crude PCR product was removed and added to a second PCR mixture containing only primers sFvA and sFvD. This second PCR synthesis also comprised 25 cycles each with a profile identical to that of the previous amplification. This product was gel purified using Geneclean II (Bio 101), digested overnight at room temperature with Nco I and Spe I, and cloned into the Novagen vector pET-22b (+).

### EXAMPLE 28

Expression of sFvZME-018 in E.coli

Positive sFvZME-018/pET-22b DNA clones were transformed into competent E. coli BL21 (DE3)pLysS and induced with varying concentrations of IPTG for different lengths of time at several temperatures in 2×YT growth medium. Final conditions chosen for expression involved induction with 0.2 mM IPTG at an A600 of approximately 0.2 for 18 hours at 30° C. Periplasmic fractions were isolated by osmotic shock and supernatants were used directly for screening using ELISA and Western analyses.

### EXAMPLE 29

Construction of the Gene Encoding scFvZME-018-Geionin Immunotoxin

The genes encoding scFvZME-018 and gelonin were linked together in a 5′ Nco I- and 3′ Hind III-flanked scFv-gelonin orientation via a short (Gly-Gly-Gly-Gly-Ser) peptide tether to provide flexibility between the two proteins in a PCR-based method similar to the one described above. Briefly, DNA encoding sFvZME-018 was amplified using the primers sFvA and 3 (5′-CCGGAGCCACCGCCACCGCTAGCTGAGGAGACTGTGA-3′). Simultaneously, DNA encoding gelonin from the vector pRCM1808B was amplified using the primers 2 (5′-GGTGGCGGTGGCTCCGGTCTAGATACCGTTAGC) and 4 (5′-GCCGCAAGCTTAATAGTTACAGCTCGTCTTT CTCGAGGAATTTCAGCAG. Aliquots of these two reactions were then mixed together and re-amplified as described above using only the primers sFvA and 4. The final 1,500 bp fragment was gel purified using Geneclean II (Bio 101), digested overnight at room temperature with Nco I and Hind III, and cloned into the Novagen vector pET-22b (+). Aliquots of these two reactions were then mixed together and re-amplified as described above using only the primers sFvA and 4.

### EXAMPLE 30

Expression of the scFvZME-018-Gelonin Immunotoxin in E. Coli

pET-22b clones encoding full-length immunotoxin, as judged by restriction digest analysis, were transformed into competent E. coli BL21 (DE3)pLysS and incubated in 2×YT growth medium at 37° C. until the A600 of the cultures was 0.4. IPTG was added to a final concentration of 1 mM and induction was continued overnight at 16° C. The periplasmic fractions of the harvested bacteria were isolated using osmotic shock and mild sonication and supernatants were used directly in ELISA and Western analyses. ELISA and Western analyses Wells of a 96-well microtiter plate were coated overnight with antibody 13A3, an anti-gelonin murine monoclonal antibody and then blocked with BSA. In subsequent steps periplasmic lysates containing immunotoxin clones, rabbit anti-gelonin polyclonal antibody, and finally horseradish peroxidase-conjugated goat anti-rabbit IgG were added. The plate was developed with ABTS and the signal quantitated at 405 nm. For Western blots, periplasmic lysates were separated by 11% SDS-PAGE and transferred onto nitrocellulose. The filters were blocked in 5% BSA and then reacted with 0.1 ug/mL of rabbit anti-gelonin polyclonal antibody in 1% BSA. After extensively washing the filters in TRIS Buffer Saline (TBS)-0.5% Tween 20, 0.1 ug/mL horseradish peroxidase-conjugated goat anti-rabbit IgG was added. Following this incubation, the filters were again washed in TBS-0.5% and Tween 20 and developed with the Amersham ECL detection system.

Cloning of sFvZME-018-Gelonin as a glutathione-S-transferase (GST) fusion protein DNA encoding the sFvZME-018-Gelonin immunotoxin from pET-22b was digested with Nco I, blunt-ended with DNA Pol I Klenow and dNTPs, and then purified using the PCR purification kit from Qiagen. The DNA was then digested with Hind III, gel purified using Geneclean II (Bio 101) and cloned into the Sma I and Hind III sites of the GST fusion vector pGEX-2T (Pharmacia). By virtue of this cloning method three extra N-terminal amino acids (Pro-Met-Ala) were added onto the antibody fragment of the immunotoxin.

### EXAMPLE 31

Expression of sFvZME-018-Gelonin as a (GST) Fusion Protein in E. coli

Positive DNA clones encoding the GST-immunotoxin fusion were transformed into the E. coli strains JM109, XL1-Blue MRF, E104, and BL21 and induced under a variety of conditions. The optimal conditions were determined to be as follows: transformed cells were grown in 2×YT at 37° C. to an A600 of 0.7-0.9 when Isopropyl-B-D-thiogalactopyranoside (IPTG) was added to a final concentration of 0.2 mM; cultures were then induced at 37° C. for 4 hours. Cells were centrifuged, washed with TBS (pH8) and frozen at −80° C. until use. Once thawed, the bacterial pellet was resuspended in TBS (pH 8), 2 mg/mL lysozyme and I mM phenylmethylsulfonofluoride (PMSF) and sonicated. Supernatants were saved and bound to Glutathione-agarose (Sigma) for 30 minutes at room temperature. The resin was then washed successively with an excess of 1M NaCl in TBS (pH 8) and TBS (pH 8). Fusion protein bound to the resin was then either eluted off the resin with 15 mm reduced glutathione (GSH) or digested directly with thrombin (Novagen) overnight at room temperature using standardized conditions.

### EXAMPLE 32

Construction and Expression of scFvZME-018

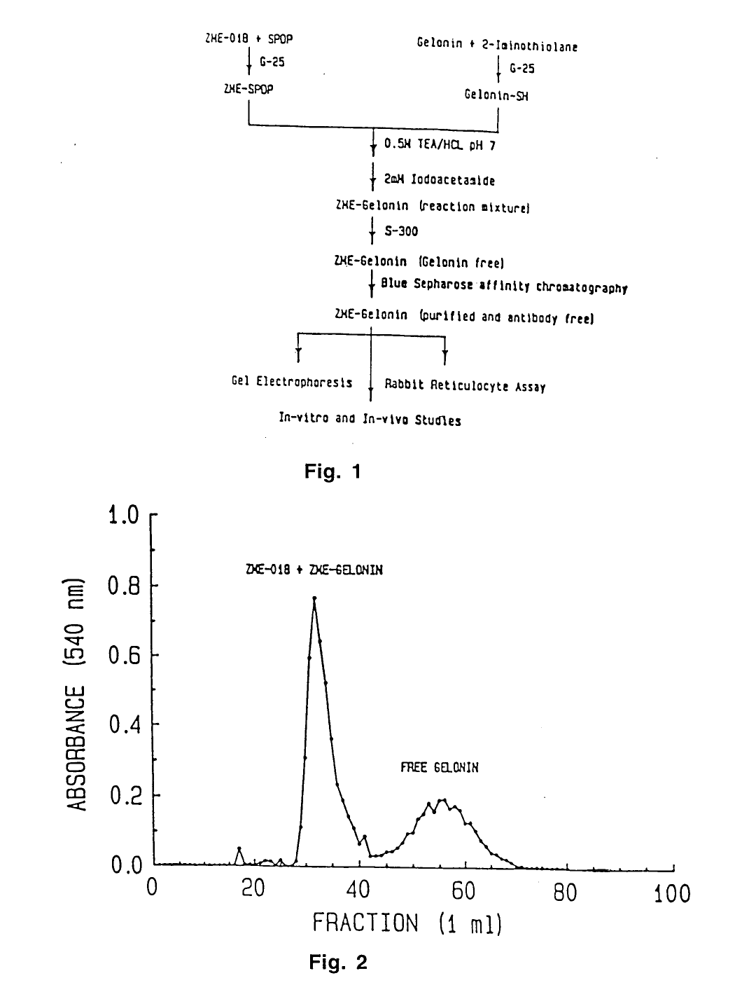

The Novagen Ig-Prime System takes advantage of the fact that mammalian antibody heavy and light chains contain conserved sequences adjacent to the immunoglobulin variable regions. Appropriately designed sets of oligonucleotide primers therefore allow specific PCR amplification of these regions following which the products are directly cloned into plasmid vectors for confirmatory DNA sequence analysis. Several hundred heavy and light chain variable region clones of antibody ZME-018 were identified and five of each were sequenced to determine the DNA sequence at the terminii of the antibody V-regions. The information obtained from this was used to design oligonucleotide primers to construct the single-chain analogue of ZME-018 which was asembled in a two-step overlap-extension PCR method. The linker chosen to tether the two antibody variable regions (and incorporated into the two primers sFvC and sFvC) was the 218 sequence (GSTSGSGKPGSGEGSTKG) which was shown to have less proteolytic susceptibility than other frequently used linkers. The predominant products of the first 25 cycles of PCR are the individual light and heavy chain variable domain-encoding segments (approximately 350 bp each) with a minor product encoding the full length 750 bp scFv gene. The second set of PCR cycles substantially enriches for full length DNA comprising the VL sequence linked to the VH region via the 18 amino acid linker (FIG. 1). This product was gel purified and then digested with Nco I and Spe I and cloned into the T7-expression vector pET-22b. Prior to construction of the scFvZME-018-Gelonin immunotoxin, the functionality of the scFv fragment was to be tested for stability and binding avidity to target hapten gp240. Several dozen positive clones were induced with IPTG and the bacterial periplasmic lysates were tested by ELISA for binding activity to gp240. Briefly, human metastatic tumor cells A375M or AAB-527 cells expressing the gp240 antigen on their cell surfaces were coated onto the bottom of the wells on a 96-well ELISA plate. Unreacted sites were subsequently blocked with BSA before addition of the bacterial supernatants. Unbound material was washed off and several different combinations of primary, secondary, and tertiary detecting antibodies were then successively added to the wells in an effort to amplify the weak ELISA signal (data not shown). This was necessitated by the fact that the target scFv antibody lacked the constant region recognition epitopes necessary for the commercially available detection antibodies to bind with sufficient avidity. Consequently, even though some bacterial lysates were weakly Western-positive for scFvZME-018 (FIG. 2), it was not possible to determine whether the expressed scFv was in fact functional or inactive. In order to be able to accurately assay the functionality and binding of the antibody clones to the hapten it became necessary to express the scFv clones as fusions with gelonin. The toxin moiety would serve as a convenient target for the several anti-gelonin antibodies available thereby facilitating the detection and quantitation of binding activity of the scFv fragment.

### EXAMPLE 33

Construction and Expression of scFvZME-018-Gelonin Immunotoxins

Gelonin was fused to the C-terminal of scFvZME-018 via a short, nonstructured Gly4Ser linker to provide a measure of flexibility between the two protein moities. The immunotoxin was assembled with the same PCR-based method used to construct the scFv fragment. The products of the first set of PCR cycles are the individual scFv antibody and toxin fragments whereas the predominant product from the second set of reactions is the full-length immunotoxin. For possible increased cytotoxicity, the endoplasmic reticulum retrieval signal KDEL was added to the C-terminal sequence of gelonin to provide for more efficient transport of the toxin to the endoplasmic reticulum from where translocation into the cytosol occurs. The final PCR product was gel purified, digested with Nco I and Hind III, and cloned into the T7-expression vector pET-22b. Bacterial clones containing full-length immunotoxin DNA were induced with IPTG and both culture supernatants and periplasmic extracts screened by ELISA for binding to both antibody-specific hapten gp240 and 13A3, an anti-gelonin murine monoclonal antibody (FIG. 3).

Functional immunotoxin bound to target was detected with a polyclonal rabbit anti-gelonin antibody followed by a horseradish peroxidase-conjugated goat anti-rabbit IgG antibody. Several immunotoxin clones with the highest binding titers to both A375M cells and antibody 13A3 were chosen for sequencing (FIG. 22) and further characterization. Different bacterial hosts and induction conditions were tested to improve expression yields and reduce the degradation problems initially observed with the immunotoxins. These manipulations were largely successful on a small scale (5 mL). Using a combination of ion-exchange, hydroxyapatite, and afinity chromatography, yields and purity of protein obtained were unviable, (data not shown) and the GST-fusion system was selected as a possible means to improve expression yields of the immunotoxin.

### EXAMPLE 34

Expression of scFvZME-018-Gelonin Immunotoxins as a GST-Fusion

The pGEX-2T vector was used to express the scFvZME-018-Gelonin immunotoxin as a glutathione-S-transferase fusion. Proteins expresses as GST fusions are generally purified in high yields using non-denaturing conditions in a one-step procedure with glutathione-agarose (GSH-ag) affinity chromatography. The vector has been designed so that the GST carrier can be cleaved from the target fusion protein by virtue of a thrombin cleavage site between the two protein moieties. Furthermore, any contaminating GST or undigested fusion protein can be removed by rebinding to GSH-ag. The incompatible 5′ restriction sites in the vector necessitated blunt-ending the 5′ end of the immunotoxin and the consequent addition of three extra amino acids (Pro-Met-Ala) to the N-terminal of scFvZME-018. However, this would not be expected to adversely affect its binding activity since this end of an antibody is far removed from the binding site. Different induction conditions were used to optimize expression of soluble fusion protein but in all cases most of the target GST-fusion was produced as cytoplasmic inclusion bodies. Any material isolated from soluble supernatants was unrecoverable from GSH-agarose due to precipitation of the protein and current work in the lab is focussing on improving the expression efficiency of soluble material.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A composition of matter comprising a conjugate of a ZME-018 antibody and a cytotoxic moiety selected from the group consisting of gelonin, recombinant gelonin, and gelonin derivatives.

2-7. (canceled)

9-14. (canceled)

15. An immunotoxin, comprising:
a single chain antibody of ZME-0 18 directed against the 240 kD antigen of gp240; and,
a cytotoxic moiety selected from the group consisting of gelonin, recombinant gelonin and a gelonin derivative.

16-18. (canceled)
